(19) United States
(12) Patent Application Publication
(10) Pub. No.: US 2003/0194745 A1
(43) Pub. Date: Oct. 16, 2003

(54) Cysteine mutants and methods for detecting ligand binding to biological molecules

(76) Inventors: Robert S. McDowell, San Francisco, CA (US); W. Michael Flanagan, Menlo Park, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/214,419

(22) Filed: Aug. 5, 2002

Related U.S. Application Data
(63) Continuation-in-part of application No. 09/981,547, filed on Oct. 17, 2001.
(62) Division of application No. 09/105,372, filed on Jun. 26, 1998, now Pat. No. 6,335,155.
(63) Continuation-in-part of application No. 09/990,421, filed on Nov. 21, 2001.
(63) Continuation-in-part of application No. 10/121,216, filed on Apr. 10, 2002.
(60) Provisional application No. 60/310,725, filed on Aug. 7, 2001.

(51) Int. Cl. G01N 33/53; G06F 19/00; G01N 33/48; G01N 33/50

(52) U.S. Cl. 435/7.1; 702/19

(57) ABSTRACT

The present invention relates generally to variants of target biological molecules (“TBMs”) and to methods of making and using the same to identify ligands of TBMs. More specifically, the invention relates to individual variant TBMs and sets of variant TBMs, each of which represents a modified version of a protein of interest where a thiol has been introduced at or near a site of interest. Ligands of TBMs are identified in part through the formation of a covalent bond between a potential ligand and a reactive thiol on the TBM.

This application asserts priority to U.S. Provisional Application No. 60/310,725 filed Aug. 7, 2001. This application is also: (a) a continuation-in-part of U.S. Ser. No. 09/981,547 filed Oct. 17, 2001 which is a divisional of U.S. Ser. No. 09/105,372 filed Jun. 26, 1998 (now U.S. Pat. No. 6,335,155); (b) a continuation-in-part of U.S. Ser. No. 09/990,421 filed Nov. 21, 2001; and (c) a continuation-in-part of U.S. Ser. No. 10/121,216 filed Apr. 10, 2002. All of these priority applications are incorporated herein by reference.

### BACKGROUND

The drug discovery process usually beings with massive functional screening of compound libraries to identify modest affinity leads (K_{d}˜1 to 10 μM) for subsequent medicinal chemistry optimization. However, not all targets of interest are amenable to such screening. In some cases, an assay that is amenable to high throughput screening is not available. In other cases, the target can have multiple binding modes such that the results of such screens are ambiguous and difficult to interpret. Still in other cases, the assay conditions for high throughput screening are such that they are prone to artifacts. As a result, alternative methods for ligand discovery are needed that to not necessarily rely on functional assays. The present invention provides such methods.

### SUMMARY

The present invention relates generally to variants of target biological molecules (“TBMs”) and to methods of making and using the same to identify ligands of TBMs. More specifically, the invention relates to individual variant TBMs and sets of variant TBMs, each of which represents a modified version of a protein of interest where a thiol has been introduced at or near a site of interest. Ligands of TBMs are identified in part through the formation of a covalent bond between a potential ligand and a reactive thiol on the TBM.

### DESCRIPTION OF THE FIGURES

FIG. 1 schematically illustrates one embodiment of the tethering method wherein the target is a protein and the covalent bond is a disulfide. A thiol-containing protein is reacted with a plurality of ligand candidates. A ligand candidate that possesses an inherent binding affinity for the target is identified and a ligand is made comprising the identified binding determinant (represented by the circle).

FIG. 2 is a representative example of a tethering experiment. FIG. 2A is the deconvoluted mass spectrum of the reaction of thymidylate synthase (“TS”) with a pool of 10 different ligand candidates with little or no binding affinity for TS. FIG. 2B is the deconvoluted mass spectrum of the reaction of TS with a pool of 10 different ligand candidates where one of the ligand candidates possesses an inherent binding affinity to the enzyme.

FIG. 3 shows three illustrative examples of the distribution pattern of the residues that are each mutated to a cysteine. FIG. 3A is an example where the residues are distributed about a single site of interest. The structure is of the core domain of HIV integrase with the portion comprising the site of interest shaded in dark gray. FIG. 3B is an example where the residues are distributed about two sites of interest. The structure is of the human interleukin-1 receptor with the portions comprising the two sites of interested shaded in dark gray. FIG. 3C is an example where the residues are distributed throughout the surface of a protein. The structure is the trimeric structure of human TNF-α.

FIG. 4 shows the side chain rotamers of cysteines in A) β-sheets and B) α-helices.

### DESCRIPTION OF THE PREFERRED EMBODIMENTS

The present invention relates generally to variants of target biological molecules (“TBMs”) and to methods of making and using the same to identify ligands of TBMs.

Unless defined otherwise, technical and scientific terms used herein have the same meaning as commonly understood by one of ordinary skill in the art to which this invention belongs. References, such as Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

Definitions

The definition of terms used herein include:

The term “aliphatic” or “unsubstituted aliphatic” refers to a straight, branched, cyclic, or polycyclic hydrocarbon and includes alkyl, alkenyl, alkynyl, cycloalkyl, cycloalkenyl, and cycloalkynyl moieties.

The term “alkyl” or “unsubstituted alkyl” refers to a saturated hydrocarbon.

The term “alkenyl” or “unsubstituted alkenyl” refers to a hydrocarbon with at least one carbon-carbon double bond.

The term “alkynyl” or “unsubstituted alkynyl” refers to a hydrocarbon with at least one carbon-carbon triple bond.

The term “aryl” or “unsubstituted aryl” refers to mono or polycyclic unsaturated moieties having at least one aromatic ring. The term includes heteroaryls that include one or more heteroatoms within the at least one aromatic ring. Illustrative examples of aryl include: phenyl, naphthyl, tetrahydronaphthyl, indanyl, indenyl, pyridyl, pyrazinyl, pyrimidinyl, pyrrolyl, pyrazolyl, imidazolyl, thiazolyl, oxazolyl, isooxazoly, thiadiazolyl, oxadiazolyl, thiophenyl, furanyl, quinolinyl, isoquinolinyl, and the like.

The term “substituted” when used to modify a moiety refers to a substituted version of the moiety where at least one hydrogen atom is substituted with another group including but not limited to: aliphatic; aryl, alkylaryl, F, Cl, I, Br, —OH; —NO₂; —CN; —CF₃; —CH₂CF₃; —CH₂Cl; —CH₂OH; —CH₂CH₂OH; —CH₂NH₂; —CH₂SO₂CH₃; —OR^{x}; —C(O)R^{x}; —COOR^{x}; —C(O)N(R^{x})₂; —OC(O)R^{x}; —OCOOR^{x}; —OC(O)N(R^{x})₂; —N(R^{x})₂; —S(O)₂R^{x}; and —NR^{x}C(O)R^{x} where each occurrence of R^{x} is independently hydrogen, substituted aliphatic, unsubstituted aliphatic, substituted aryl, or unsubstituted aryl. Additionally, substitutions at adjacent groups on a moiety can together form a cyclic group.

The term “antagonist” is used in the broadest sense and includes any ligand that partially or fully blocks, inhibits or neutralizes a biological activity exhibited by a target, such as a TBM. In a similar manner, the term “agonist” is used in the broadest sense and includes any ligand that mimics a biological activity exhibited by a target, such as a TBM, for example, by specifically changing the function or expression of such TBM, or the efficiency of signaling through such TBM, thereby altering (increasing or inhibiting) an already existing biological activity or triggering a new biological activity.

The term “ligand” refers to an entity that possesses a measurable binding affinity for the target. In general, a ligand is said to have a measurable affinity if it binds to the target with a K_{d} or a Kₗ of less than about 100 mM, preferably less than about 10 mM, and more preferably less than about 1 mM. In preferred embodiments, the ligand is not a peptide and is a small molecule. A ligand is a small molecule if it is less than about 2000 daltons in size, usually less than about 1500 daltons in size. In more preferred embodiments, the small molecule ligand is less than about 1000 daltons in size, usually less than about 750 daltons in size, and more usually less than about 500 daltons in size.

The term “ligand candidate” refers to a compound that possesses or has been modified to possess a reactive group that is capable of forming a covalent bond with a complimentary or compatible reactive group on a target. The reactive group on either the ligand candidate or the target can be masked with, for example, a protecting group.

The term “polynucleotide”, when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The phrase “protected thiol” as used herein refers to a thiol that has been reacted with a group or molecule to form a covalent bond that renders it less reactive and which may be deprotected to regenerate a free thiol.

The phrase “reversible covalent bond” as used herein refers to a covalent bond that can be broken, preferably under conditions that do not denature the target. Examples include, without limitation, disulfides, Schiff-bases, thioesters, coordination complexes, boronate esters, and the like.

The phrase “reactive group” is a chemical group or moiety providing a site at which a covalent bond can be made when presented with a compatible or complementary reactive group. Illustrative examples are —SH that can react with another —SH or —SS— to form a disulfide; an —NH₂ that can react with an activated —COOH to form an amide; an —NH₂ that can react with an aldehyde or ketone to form a Schiff base and the like.

The phrase “reactive nucleophile” as used herein refers to a nucleophile that is capable of forming a covalent bond with a compatible functional group on another molecule under conditions that do not denature or damage the target. The most relevant nucleophiles are thiols, alcohols, and amines. Similarly, the phrase “reactive electrophile” as used herein refers to an electrophile that is capable of forming a covalent bond with a compatible functional group on another molecule, preferably under conditions that do not denature or otherwise damage the target. The most relevant electrophiles are imines, carbonyls, epoxides, aziridies, sulfonates, disulfides, activated esters, activated carbonyls, and hemiacetals.

The phrase “site of interest” refers to any site on a target on which a ligand can bind. For example, when the target is an enzyme, the site of interest can include amino acids that make contact with, or lie within about 10 Angstroms (more preferably within about 5 Angstroms) of a bound substrate, inhibitor, activator, cofactor, or allosteric modulator of the enzyme. When the enzyme is a protease, the site of interest includes the substrate binding channel from S6 to S6′, residues involved in catalytic function (e.g. the catalytic triad and oxy anion hole), and any cofactor (e.g. metal such as Zn) binding site. When the enzyme is a protein kinase, the site of interest includes the substrate-binding channel in addition to the ATP binding site. When the enzyme is a dehydrogenease, the site of interest includes the substrate binding region as well as the site occupied by NAD/NADH. When the enzyme is a hydralase such as PDE4, the site of interest includes the residues in contact with cAMP as well as the residues involved in the binding of the catalytic divalent cations.

The terms “target,” “Target Molecule,” and “TM” are used interchangeably and in the broadest sense, and refer to a chemical or biological entity for which the binding of a ligand has an effect on the function of the target. The target can be a molecule, a portion of a molecule, or an aggregate of molecules. The binding of a ligand may be reversible or irreversible. Specific examples of target molecules include polypeptides or proteins such as enzymes and receptors, transcription factors, ligands for receptors such growth factors and cytokines, immunoglobulins, nuclear proteins, signal transduction components (e.g., kinases, phosphatases), polynucleotides, carbohydrates, glycoproteins, glycolipids, and other macromolecules, such as nucleic acid-protein complexes, chromatin or ribosomes, lipid bilayer-containing structures, such as membranes, or structures derived from membranes, such as vesicles. The definition specifically includes Target Biological Molecules (“TBMs”) as defined below.

A “Target Biological Molecule” or “TBM” as used herein refers to a single biological molecule or a plurality of biological molecules capable of forming a biologically relevant complex with one another for which a small molecule agonist or antagonist has an effect on the function of the TBM. In a preferred embodiment, the TBM is a protein or a portion thereof or that comprises two or more amino acids, and which possesses or is capable of being modified to possess a reactive group that is capable of forming a covalent bond with a compound having a complementary reactive group. Preferred TBMs include: cell surface and soluble receptors and their ligands; steroid receptors; hormones; immunoglobulins; clotting factors; nuclear proteins; transcription factors; signal transduction molecules; cellular adhesion molecules, co-stimulatory molecules, chemokines, molecules involved in mediating apoptosis, enzymes, and proteins associated with DNA and/or RNA synthesis or degradation.

Many TBMs are those participate in a receptor-ligand binding interaction and can be either member of a receptor-ligand pair. Illustrative examples of growth factors and their respective receptors include those for: erythropoietin (EPO), thrombopoietin (TPO), angiopoietin (ANG), granulocyte colony stimulating factor (G-CSF), granulocyte macrophage colony stimulating factor (GM-CSF), epidermal growth factor (EGF), heregulin-α and heregulin-β, vascular endothelial growth factor (VEGF), placental growth factor (PLGF), transforming growth factors (TGF-α and TGF-β), nerve growth factor (NGF), neurotrophins, fibroblast growth factor (FGF), platelet-derived growth factor (PDGF), bone morphogenetic protein (BMP), connective tissue growth factor (CTGF), hepatocyte growth factor (HGF), and insulin-like growth factor 1 (IGF-1). Illustrative examples of hormones and their respective receptors include those for: growth hormone, prolactin, placental lactogen (LPL), insulin, follicle stimulating hormone (FSH), luteinizing hormone (LH), and neurokinin-1. Illustrative examples of cytokines and their respective receptors include those for: ciliary neurotrophic factor (CNTF), oncostatin M (OSM), TNF-α; CD40L, stem cell factor (SCF); interleukin-1, interleukin-2, interleukin-4, interleukin-5, interleukin-6, interleukin-8, interleukin-9, interleukin-13, and interleukin-18.

Other TBMs include: cellular adhesion molecules such as CD2, CD11a, LFA-1, LFA-3, ICAM-5, VCAM-1, VCAM-5, and VLA-4; costimulatory molecules such as CD28, CTLA-4, B7-1; B7-2, ICOS, and B7RP-1; chemokines such as RANTES and MIP1b; apoptosis factors such as APAF-1, p53, bax, bak, bad, bid, and c-ab1; anti-apoptosis factors such as bc12, bc1-x(L), and mdm2; transcription modulators such as AP-1 and AP-2; signaling proteins such as TRAF-1, TRAF-2, TRAF-3, TRAF-4, TRAF-5, and TRAF-6; and adaptor proteins such as grb2, cb1, shc, nck, and crk

Enzymes are another class of preferred TBMs and can be categorized in numerous ways including as: allosteric enzymes; bacterial enzymes (isoleucyl tRNA synthase, peptide deformylase, DNA gyrase, and the like); fungal enzymes (thymidylate synthase and the like); viral enzymes (HIV integrase, HSV protease, Hepatitis C helicase, Hepatitis C protease, rhinovirus protease and the like); kinases (serine/threonine, tyrosine, and dual specificity); phosphatases (serine/threonine, tyrosine, and dual specificity); and proteases (aspartyl, cysteine, metallo, and serine proteases). Notable subclasses of enzymes include: kinases such as Lck, Syk, Zap-70, JAK, FAK, ITK, BTK, MEK, MEKK, GSK-3, Raf, tgf-β-activated kinase-1 (TAK-1), PAK-1, cdk4, Akt, PKC θ, IKK β, IKK-2, PDK, ask, nik, MAPKAPK, p90rsk, p70s6k, and P13-K (p85 and p110 subunits); phosphatases such as CD45, LAR, RPTP-α, RPTP-μ, Cdc25A, kinase-associated phosphatase, map kinase phosphatase-1, PTP-1B, TC-PTP, PTP-PEST, SHP-1 and SHP-2; caspases such as caspases-1, -3, -7, -8, -9, and -11; and cathespins such as cathepsins B, F, K, L, S, and V. Other enzymatic targets include: BACE, TACE, cytosolic phospholipase A2 (cPLA2), PARP, PDE I-VII, Rac-2, CD26, inosine monophosphate dehydrogenase, 15-lipoxygenase, acetyl CoA carboxylase, adenosylmethionine decarboxylase, dihydroorotate dehydrogenase, leukotriene A4 hydrolase, and nitric oxide synthase.

Variants of TBMs

The present invention relates generally to variants of target biological molecules (“TBMs”) and to methods of making and using the same to identify ligands of the TBMs. In preferred embodiments, the TBMs are proteins and the variants are cysteine mutants thereof wherein a naturally occurring non-cysteine residue of a TBM is mutated into a cysteine residue. The non-native cysteine provides a reactive group on the TBM for use in tethering.

Tethering is a method of ligand identification that relies upon the formation of a covalent bond between a reactive group on a target and a complimentary reactive group on a potential ligand, and is described in U.S. Pat. No. 6,335, 155, PCT Publication Nos. WO 00/00823 and WO 02/42773, Erlanson et al., Proc. Nat. Acad. Sci. USA 97: 9367-9372 (2000), and U.S. Ser. No. 10/121,216 entitled METHODS FOR LIGAND DISCOVERY by inventors Daniel Erlanson, Andrew Braisted, and James Wells (corresponding PCT Application No. US02/13061), all of which are incorporated herein by reference. The resulting covalent complex is termed a target-ligand conjugate. Because the covalent bond is formed at a pre-determined site on the target (e.g., a native or non-native cysteine), the stoichiometry and binding location are known for ligands that are identified by this method.

Once formed, the ligand portion of the target-ligand conjugate can be identified using a number of methods. In preferred embodiments, mass spectroscopy is used. The target-ligand can be detected directly in the mass spectrometer or fragmented prior to detection. Alternatively, the ligand can be liberated from the target-ligand conjugate within the mass spectrophotometer and subsequently identified. In other embodiments, alternate detection methods are used including to but not limited to: chromatography, labeled probes (fluorescent, radioactive, etc.), nuclear magnetic resonance (“NMR”), surface plasmon resonance (e.g., BIACORE), capillary electrophoresis, X-ray crystallography and the like. In still other embodiments, functional assays can also be used when the binding occurs in an area essential for what the assay measures.

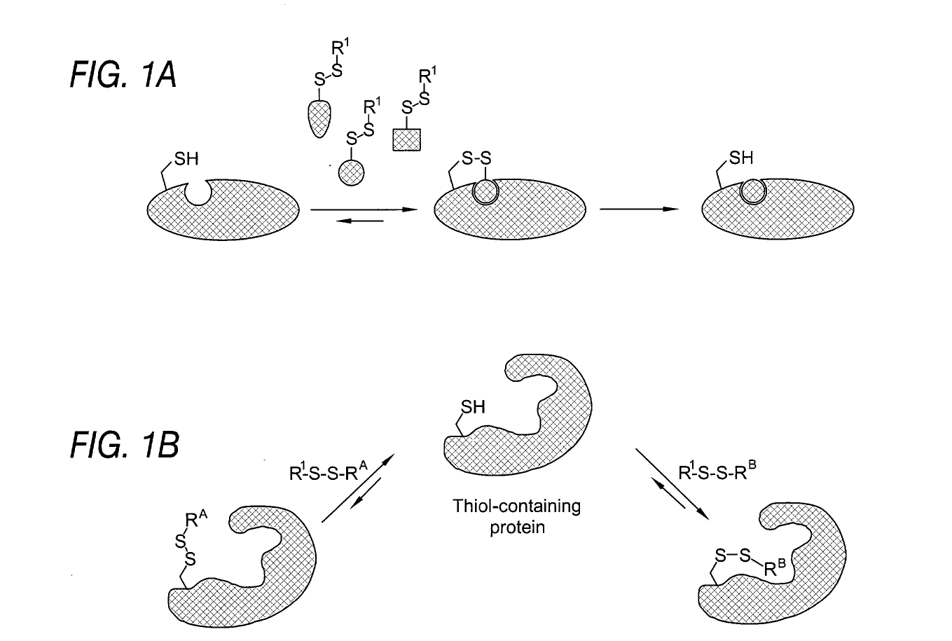

A schematic representation of one embodiment of the tethering method where the target is a protein and the covalent bond is a disulfide is shown in FIG. 1. A thiol containing protein is reacted with a plurality of ligand candidates. In this embodiment, the ligand candidates possess a masked thiol in the form of a disulfide of the formula —SSR¹ where R¹ is unsubstituted C₁-C₁₀ alkyl, substituted C₁-C₁₀ alkyl, unsubstituted aryl or substituted aryl. In certain embodiments, R¹ is selected to enhance the solubility of the potential ligand candidates. As shown, a ligand candidate that possesses an inherent binding affinity for the target is identified and a corresponding ligand that does not include the disulfide moiety is made comprising the identified binding determinant (represented by the circle).

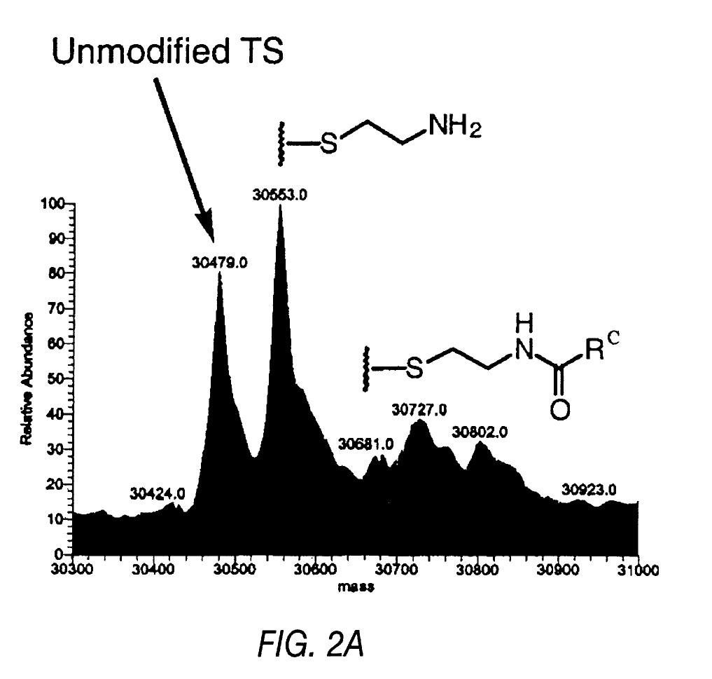

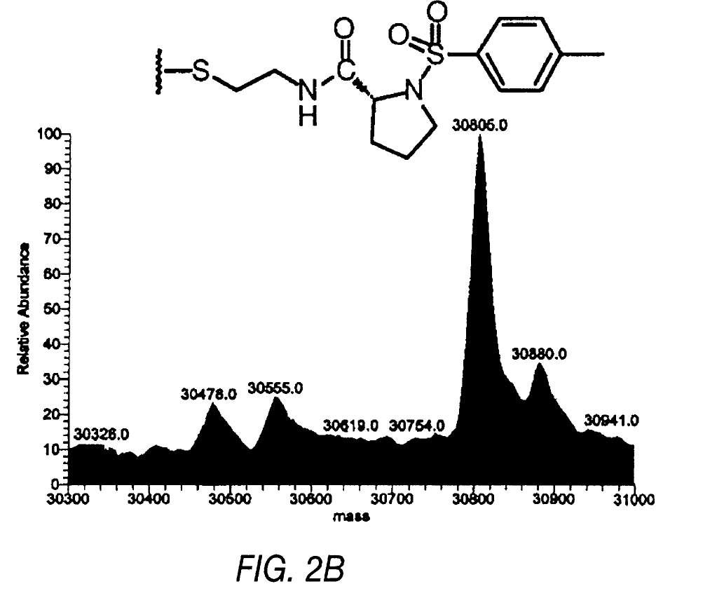

FIG. 2 illustrates two representative tethering experiments where a target enzyme, E. coli thymidylate synthase, is contacted with ligand candidates of the formula

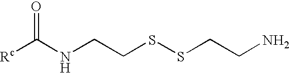

wherein R^{c} is the variable moiety among this pool of library members and is unsubstituted aliphatic, substituted aliphatic, unsubstituted aryl, or substituted aryl. Like all TS enzymes, E. coli TS has an active site cysteine (Cys146) that can be used for tethering. Although the E. coli TS also includes four other cysteines, these cysteines are buried and were found not to be reactive in tethering experiments. For example, in an initial experiment, wild type E. coli TS and the C146S mutant (wherein the cysteine at position 146 has been mutated to serine) were contacted with cystamine, H₂NCH₂CH₂SSCH₂CH₂NH₂. The wild type TS enzyme reacted cleanly with one equivalent of cystamine while the mutant TS did not react indicating that the cystamine was reacting with and was selective for Cys146.

FIG. 2A is the deconvoluted mass spectrum of the reaction of TS with a pool of 10 different ligand candidates with little or no binding affinity for TS. In the absence of any binding interactions, the equilibrium in the disulfide exchange reaction between TS and an individual ligand candidate is to the unmodified enzyme. This is schematically illustrated by the following equation.

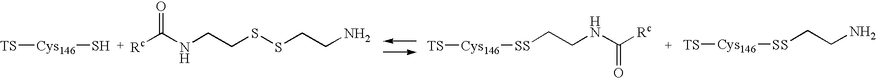

As expected, the peak that corresponds to the unmodified enzyme is one of two most prominent peaks in the spectrum. The other prominent peak is TS where the thiol of Cys146 has been modified with cysteamine. Although this species is not formed to a significant extent for any individual library member, the peak is due to the cumulative effect of the equilibrium reactions for each member of the library pool. When the reaction is run in the presence of a thiol-containing reducing agent such as 2-mercaptoethanol, the active site cysteine can also be modified with the reducing agent. Because cysteamine and 2-mercaptoethanol have similar molecular weights, their respective disulfide bonded TS enzymes are not distinguishable under the conditions used in this experiment. The small peaks on the right correspond to discreet library members. Notably, none of these peaks are very prominent. FIG. 2A is characteristic of a spectrum where none of the ligand candidates possesses an inherent binding affinity for the target.

FIG. 2B is the deconvoluted mass spectrum of the reaction of TS with a pool of 10 different ligand candidates where one of the ligand candidates possesses an inherent binding affinity to the enzyme. As can be seen, the most prominent peak is the one that corresponds to TS where the thiol of Cys146 has been modified with the N-tosyl-D-proline compound. This peak dwarfs all others including those corresponding to the unmodified enzyme and TS where the thiol of Cys146 has been modified with cysteamine. FIG. 2B is an example of a mass spectrum where tethering has captured a moiety that possesses a strong inherent binding affinity for the desired site.

The representative tethering experiments of FIG. 2 were performed on a TBM that already possessed a naturally occurring cysteine at a site of interest (Cys146 located in the active site of the E. coli TS enzyme). However, because TBMs do not always possess a naturally occurring cysteine at or near a site of interest, the present invention provides cysteine mutant variants of TBMs as well as methods for making the same.

Thus, in one aspect of the present invention, a set comprising at least one cysteine mutant of a protein TBM is provided wherein a naturally occurring non-cysteine residue at or near a site of interest is mutated to a cysteine residue. In one embodiment, the set comprises a plurality of cysteine mutants of a protein TBM wherein each mutant has a different naturally occurring non-cysteine residue that is mutated to a cysteine residue. In another embodiment, the set comprises at least three cysteine mutants of a protein TBM wherein each mutant has a different naturally occurring non-cysteine residue that is mutated to a cysteine residue. In yet another embodiment, the set comprises at least five cysteine mutants of a protein TBM wherein each mutant has a different naturally occurring non-cysteine residue that is mutated to a cysteine residue. In still yet another embodiment, the set comprises at least ten cysteine mutants of a protein TBM wherein each mutant has a different naturally occurring non-cysteine residue that is mutated to a cysteine residue.

In another aspect of the present invention, methods are provided for identifying residues that are suitable for mutating into cysteines. In preferred embodiments, a model or an experimentally derived three-dimensional structure (e.g., X-ray or 3D NMR) of a TBM is used to help identify residues that are suitable for mutating into cysteines. If a structure of the TBM of interest in unavailable, then a three-dimensional structure of a related or homologous TBM can be used as a stand-in. Once suitable residues are identified using the stand-in structure, then methods known in the art, such as sequence alignment, are used to identify the corresponding residues in the TBM of interest. In general, the methods described below for identifying suitable residues for mutating into cysteines can be used alone or in any combination with each other.

In one method, the local backbone conformation of a candidate residue is determined and a database of experimentally solved structures is searched for examples of a disulfide-bonded cysteine having the same or similar local backbone conformation as the candidate residue. Any combination of a residue's backbone atoms (N, C_{α}, C and O) can be used to determine the local conformation. The likelihood that the TBM accepts the cysteine mutation improves as more examples are found in a database of known disulfide-bonded cysteines in the same or similar local backbone conformation. Experimentally solved structures are available from many sources including the Protein Databank (“PDB”) which can be found on the Internet at http://www.rcsb.or and the Protein Structure Database which can be found on the Internet at http://www.pcs.com. Lists of unique, high-resolution protein chains (grouped by structures having a certain resolution and R-factor) that can be used to compile a database of experimentally solved structures are found on the Internet at http://www.fccc.edu/research/labs/dunbrack/culledpdb.html. In general, the local environment of a candidate residue includes the candidate residue itself and at least one residue preceding or following the candidate residue in sequence. A conformation is considered the same or similar if the root mean square deviation (“RMSD”) of the atoms being compared is less than or equal to about 1 Angstrom², more preferably, less than or equal to about 0.75 Angstrom², and even more preferably, less than or equal to about 0.5 Angstrom².

In one embodiment, the method comprises:

a) obtaining a set of coordinates of a three dimensional structure of a protein TBM having n number of residues;

b) selecting a candidate residue i on the three dimensional structure of the TBM wherein the candidate residue i is the ith residue where i is a number between 1 and n and residue i is not a cysteine;

c) selecting a residue j where residue j is adjacent to residue i in sequence;

d) determining a candidate reference value wherein the candidate reference value is a spatial relationship between residue i and residue j;

e) obtaining a database comprising sets of coordinates of disulfide-containing protein fragments wherein each fragment comprises at least a disulfide-bonded cysteine and a first adjacent residue where the disulfide-bonded cysteine and the first adjacent residue share the same sequential relationship as residue i and residue j;

f) determining a comparative reference value for each fragment wherein the comparative reference value is the corresponding spatial relationship between the disulfide-bonded cysteine and the first adjacent residue as the candidate reference value is between residue i and j; and,

g) determining a score wherein the score is a measure of the number of fragments in the database that possess a comparative reference value that is the same or similar to the candidate reference value.

In another embodiment, the method further comprises

selecting a residue k where residue k is adjacent to residue i in sequence and k is not j; and

wherein

the candidate reference value is a spatial relationship between residue i, residue j, and residue k;

each fragment comprises at least a disulfide-bonded cysteine, a first adjacent residue, and a second adjacent residue where the disulfide-bonded cysteine and the first and second adjacent residues share the same sequential relationship as residue i, residue j, and residue k; and

the comparative reference value is the corresponding spatial relationship between the disulfide bonded cysteine, the first adjacent residue, and the second adjacent residue as the candidate reference value is between residue i, residue j, and residue k.

In another embodiment, the method comprises:

a) obtaining a set of coordinates of a three dimensional structure of a protein TBM having n number of residues;

b) selecting a candidate residue i on the three dimensional structure of the TBM wherein the candidate residue i is the ith residue where i is a number between 1 and n and residue i is not a cysteine;

c) selecting residue j and residue k wherein residue j and residue k are both adjacent in sequence to residue i;

d) determining a candidate reference value wherein the candidate reference value is a spatial relationship of at least one backbone atom from each of residue i, residue j, and residue k;

e) obtaining a database comprising sets of coordinates of disulfide-containing protein fragments wherein each fragment comprises at least a disulfide-bonded cysteine, a first adjacent residue, and a second adjacent residue where the disulfide-bonded cysteine, the first adjacent residue, and the second adjacent residue share the same sequential relationship as residue i, residue j, and residue k;

f) determining a comparative reference value for each fragment wherein the comparative reference value is the corresponding spatial relationship between the disulfide-bonded cysteine, the first adjacent residue, and the second adjacent residue as the candidate reference value is between residue i, residue j, and residue k; and,

g) determining a score wherein the score is a measure of the number of fragments in the database that possess a comparative reference value that is the same or similar to the candidate reference value.

In another embodiment the spatial relationship comprises a dihedral angle. In yet another embodiment, the spatial relationship comprises a pair of phi psi angles. In another embodiment, the spatial relationship comprises a distance between atoms of two residues. An illustrative example of a computer algorithm for identifying disulfide bonded pairs in a database such as the PDB and matching them with a residue that is a candidate for cysteine mutation is described in Example 1.

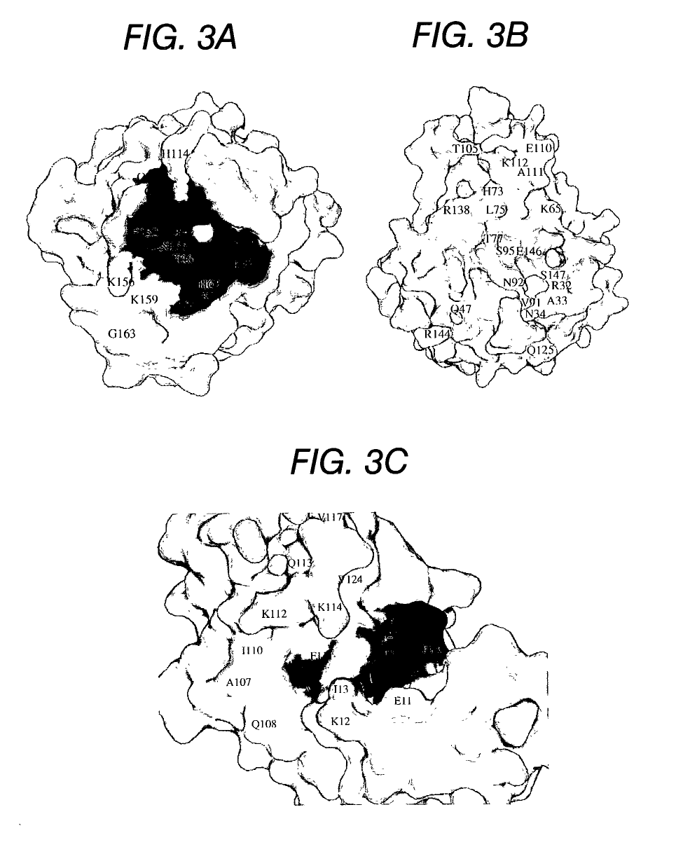

In another method, a site of interest is defined on a TBM and suitable residues for cysteine mutation are identified based on the location of the residue from the site of interest. In one embodiment, a suitable residue is a non-cysteine residue that is located within the site of interest. In another embodiment, a suitable residue is a non-cysteine residue that is located within about 5 Å from the site of interest. In yet another embodiment, a suitable residue is a non-cysteine residue that is located within about 10 Å from the site of interest. For the purposes of these measurements, any non-cysteine residue having at least one atom falling within about 5 Å or about 10 Å respectively from any atom of an amino acid within the site of interest is a suitable residue for mutating into a cysteine. A TBM can have one or multiple sites of interests. In some cases, a TBM has one site of interest and the set of residues that are each being mutated to a cysteine is clustered around this site of interest. In other cases, a TBM has at least two different sites of interest and the set of residues that are each being mutated to a cysteine is clustered around the at least two different sites of interest. Still in other cases, a TBM either does not possess a distinct site of interest or possesses multiple sites of interests such that the set of residues that are being mutated to a cysteine is dispersed throughout the protein surface. FIG. 3 shows three illustrative examples of the distribution pattern of the residues that are each mutated to a cysteine

In another method, solvent accessibility is calculated for each non-cysteine residue of a TBM and used to identify suitable residues for cysteine mutation. Solvent accessibility can be calculated using any number of known methods including using standard numeric methods (Lee, B. & Richards, F. M. J. Mol. Biol 55:379-400 (1971); Shrake, A. & Rupley, J. A. J. Mol. Biol. 79:351-371 (1973)) and analytical methods (Connolly, M. L. Science 221:709-713 (1983); Richmond, T. J. J. Mol. Biol. 178:63-89 (1984)). In one embodiment, suitable residues for mutation include residues where the combined surface area of the residue's atoms is equaled to or greater than about 20 Å². In another embodiment, suitable residues for mutation include residues where the combined surface area of the residue's atoms is equaled to or greater than about 30 Å². In yet another embodiment, suitable residues for mutation include residues where the combined surface area of the residue's atoms is equaled to or greater than about 40 Å².

In another method, suitable residues for cysteine mutation are identified by hydrogen bond analysis. In one embodiment, a suitable residue is a non-cysteine residue that does not participate in any hydrogen bond interaction. In another embodiment, a suitable residue is a non-cysteine residue whose side chain does not participate in any hydrogen bond interaction. In yet another embodiment, a suitable residue is a non-cysteine residue whose side chain does not participate in a hydrogen bond interaction with a backbone atom.

In another method, suitable residues for cysteine mutation are identified by rotamer analysis. In one embodiment, the method comprises:

a) obtaining a three dimensional structure of a TBM having n number of residues and a site of interest;

b) selecting a candidate residue i that is at or near the site of interest wherein the candidate residue i is the ith residue where i is a number between 1 and n and residue i is not a cysteine;

c) generating a set of mutated TBM structures wherein each mutated TBM structure possesses a cysteine residue instead of residue i and wherein the cysteine residue is placed in a standard rotamer conformation; and,

d) evaluating the set of mutated TBM structures.

In another embodiment, a standard rotamer conformation for cysteine comprises the set of cysteine rotamers enumerated by Ponders and Richards as described by Ponder, J. W. and Richards, F. M. J. Mol. Biol. 193: 775-791 (1987).

In another embodiment, a standard rotamer conformation for cysteine comprises a chi1 angle selected from the group consisting of about 60°, about 180°, and about 300° and a chi2 angle selected from the group consisting of about 60°, about 120°, about 180°, about 270°, and about 300°.

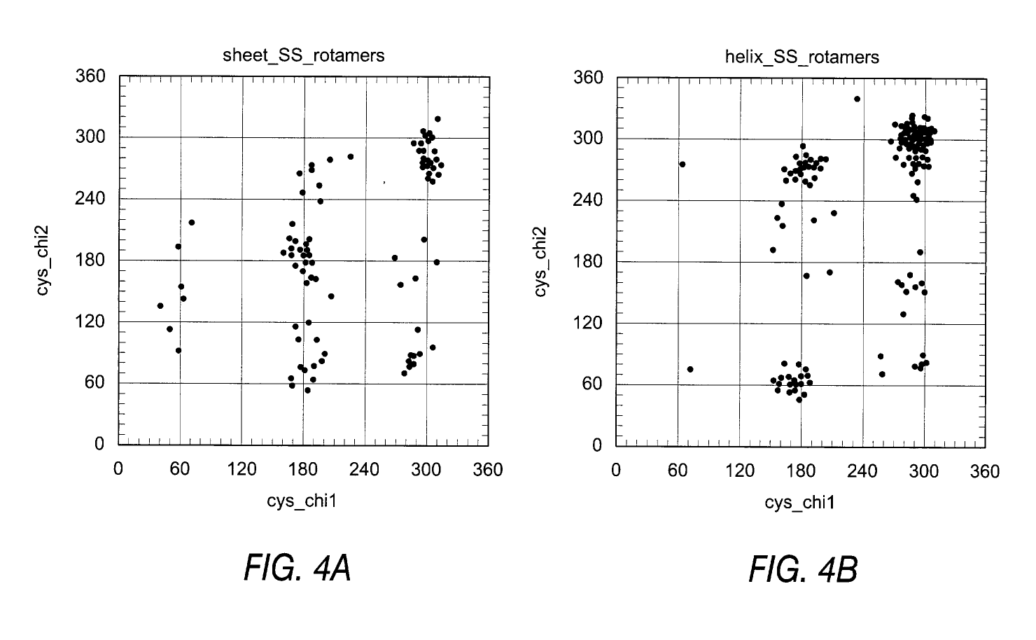

In another embodiment, the method further comprises determining whether residue i is part of an α-helix or a β-sheet and then selecting a standard rotamer conformation based on the assigned secondary structure. As shown in FIG. 4, a different set of rotamers is preferred depending on the secondary structure that is assigned to the cysteine. Residue i is considered to be part of an α-helix if the phi psi angles of residues i−1, i, and i+1 are about 300±30° and 315±30° respectively, and is considered to be part of a β-sheet if the phi psi angles of residues i−1, i, and i+1 are about 240±30° and 120±30°. If residue i is part of an α-helix, then a standard rotamer conformation for cysteine comprises a chi1 chi2 pair selected from the group consisting of about 180° and about 60°; about 180° and about 270°; and about 300° and about 300°. If residue i is part of an β-helix, then a standard rotamer conformation for cysteine comprises a chi1 chi2 pair selected from the group consisting of about 180° and about 60°; about 180° and about 180°; about 180° and about 270°; and about 300° and about 300°.

In another embodiment, the set of mutated TBM structures are evaluated based upon whether an unfavorable steric contact is made. A residue is considered to be a suitable candidate for cysteine mutation if it can be substituted with at least one cysteine rotamer for which no unfavorable steric contact is made. An unfavorable steric contact is defined as interatomic distances that are less than about 80% of the sum of the van der Waals radii of the participating atoms. In one variation, only the backbone atoms of the TBM are considered for the purposes of determining whether the rotamers make an unfavorable contact with the TBM. In another variation, the backbone atoms and C_{β} of the TBM are considered for the purposes of determining whether the rotamers make an unfavorable contact with the TBM.

In another embodiment, the set of mutated TBM structures are evaluated based on a force field calculation. Illustrative force field methods are described by, for example, Weiner, S. J. et al. J. Comput. Chem. 7: 230-252 (1986); Nemethy, G. et al. J. Phys. Chem. 96: 6472-6484 (1992); and Brooks, B. R. et al. J. Comput. Chem. 4: 187-217 (1983). All minimized conformations within about 10 kcal/mol or more preferably within about 5 kcal/mol, of the lowest-energy conformation are considered accessible.

In another embodiment, each mutated TBM structure possesses a cysteine that is capped with a S-methyl group (side chain is —CH₂SSCH₃) instead of residue i and wherein the capped cysteine residue is placed in a standard rotamer conformation for cysteine. A residue is considered to be a suitable candidate for cysteine mutation if it can be substituted with at least one rotamer that places the methyl carbon of the S-methyl group closer to the site of interest than the C_{β}

In addition to adding one or more cysteines to a site of interest, it may be desirable to delete one or more naturally occurring cysteines (and replacing them with alanines for example) that are located outside of the site of interest. These mutants wherein one or more naturally occurring cysteines are deleted or “scrubbed” comprise another aspect of the present invention. Various recombinant, chemical, synthesis and/or other techniques can be employed to modify a target such that it possesses a desired number of free thiol groups that are available for tethering. Such techniques include, for example, site-directed mutagenesis of the nucleic acid sequence encoding the target polypeptide such that it encodes a polypeptide with a different number of cysteine residues. Particularly preferred is site-directed mutagenesis using polymerase chain reaction (PCR) amplification (see, for example, U.S. Pat. No. 4,683,195 issued Jul. 28, 1987; and Current Protocols In Molecular Biology, Chapter 15 (Ausubel et al., ed., 1991). Other site-directed mutagenesis techniques are also well known in the art and are described, for example, in the following publications: Ausubel et al., supra, Chapter 8; Molecular Cloning: A Laboratory Manual., 2nd edition (Sambrook et al., 1989); Zoller et al., Methods Enzymol. 100:468-500 (1983); Zoller & Smith, DNA 3:479-488 (1984); Zoller et al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene, 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

The invention is further illustrated by the following, non-limiting examples. Unless otherwise noted, all the standard molecular biology procedures are performed according to protocols described in (Molecular Cloning: A Laboratory Manual, vols. 1-3, edited by Sambrook, J., Fritsch, E. F., and Maniatis, T., Cold Spring Harbor Laboratory Press, 1989; Current Protocols in Molecular Biology, vols. 1-2, edited by Ausbubel, F., Brent, R., Kingston, R., Moore, D., Seidman, J. G., Smith, J., and Struhl, K., Wiley Interscience, 1987).

### EXAMPLE 1

This example provides an illustrative computer algorithm written in FORTRAN for identifying disulfide pairs from the PDB that align with potential tethering mutants. A stepwise description of the program and the source code are described below.

First, a user supplies the name of the PDB file for the template protein, the residues of the fragment to match, and the relative position of the cysteine within that fragment. Preferred values are 1-2 residues N- and C-terminal to a potential mutant site. For example, if residue Glu 200 of PTP1B is a candidate residue, then the user would specify the fragment from residues 198 to 202 with the cysteine at relative position 3.

Second, the program reads the template file, extracts the coordinates of the N,C_{α},C,O atoms for the template residues, and determines the values of Φ(C′—N—C_{α}—C torsion) and ψ(N—C_{α}—C—N′) for each of the template residues

Third, the program generates a “residue filter” based on the template Φ/ψ values. This filter is used to identify contiguous segments of a test protein that have Φ/ψ values matching those of the template residues to within a coarse (±60°) tolerance. The filter also requires that the fragment must contain a cysteine at the appropriate position. In the PTP1B example above, the filter would identify 5-residue fragments of a test protein that roughly matched the backbone conformations of residues 198-202 of PTP1B and contained a cysteine in position 3.

Fourth, the rest of the program operates iteratively on a user-supplied list of test proteins provided in a simple text file. In one embodiment, this file contains approx. 2500 culled PDB chains. For each test structure:

a) The program reads the coordinates, determines the sequence and Φ/ψ values for each residue, and identifies any contiguous chains that match the residue filter specified in step (3).

b) The program checks to see that the cysteine residue in this fragment is participating in a disulfide bond. This is done by simple distance-and angle-based searching from the S_{γ} atom. Fragments containing unpaired cysteines are rejected.

c) For each fragment, the N,C_{α},C,O atoms of the backbone are overlaid onto the corresponding atoms from the template molecule (e.g. 198-202 of PTP1B). If the backbone fits with an RMSD within a user-specified tolerance (typically 0.5-0.75 Å), the overlaid coordinates of this fragment along with its disulfide-bound partner are written to a file in PDB format. A log file is maintained of each “hit”, along with its RMSD value. The hits are viewed with a graphic program like Insight II or PyMOL.

Source Code

```
p
c
  parameter(MAX_HITS = 10000)
c
$INCLUDE tk.inc
$INCLUDE tk_functions.inc
$INCLUDE rsm.inc
$INCLUDE rsm_functions.inc
c
  Record /hndl_rec/ data_handle, fragment_handle,
template_handle
  Record /atom_rec/ AtomRec
  Record /res_rec/ ResRec
  Record /res_filter/ FragmentFilter(MAX_RMS_ATOMS),
TemplateFilter(MAX_RMS_ATOMS)
  Record /vec/ TemplateVecArray, FragmentVecArray, T1, T2
  Dimension TemplateVecArray(MAX_RMS_ATOMS),
FragmentVecArray(MAX_RMS_ATOMS)
c
  Integer*4 numTemplateRes, TemplateResList(MAX_HITS),
  numHitRes,
HitResList (MAX_HITS), numTemplateVec,
  . CysIndex, FrameIndex, numSS, SS_1(MAX_RES),
  SS_2(MAX_RES),
min_element, max_element, num_res,
  . icnt, jcnt, numFragAtom, FragAtomList(MAX_RES),
FragAtomIndex(MAX_RES),ires, jres, icys, cys_idx, jcys,
  . iatom, jatom, LISTin, PDBout, LOGout, len_name, len_root
    Real*8 temp_min, temp_max, R2(3, 3), RMS_cutoff, RMS_value,
RMS_WT(MAX_RES), angle_tol
  Character listfile*80, full_name*80, file_path*80, file_name*80,
file_root*80, file_ext*80,
  . structure_name*15, full_structure_name*23, first_resnumber*7,
char1*1, char3*1, tline*80,
  . token*80
c
  LISTin = 9
  PDBout = 10
  LOGout = 11
  FrameIndex = 1
  RMS_cutoff = 0.5
  angle_tol = 60.
  do ires = 1, MAX_RES
    RMS_WT(ires) = 1.0
  end do
c
c...Get template information.
c
  write (6,‘(/,‘‘Enter template PDB filename : ’’,$)’)
  read (5,‘(a)’) tline
  if (.not.readPDBFile (tline, template_handle)) then
    write (6,‘(‘‘ERROR: Unable to read template PDB file
***’’)’)
    return
  end if
  if (get_num_total_residues(template_handle, num_res)) continue
c...get template residue numbers and convert to residue indeces
10 write (6,‘(5x,‘‘Enter beginning, ending template residues :
’’,$)’)
  read (5,‘(a)’) tline
  if (.not.get_token(tline, token)) goto 10
  do icnt = 1, num_res
    if (getResData(template_handle, FrameIndex, icnt, ResRec))
continue
    if (ljust(ResRec.residue_number)) continue
    if (compstr (ResRec.residue_number, token)) then
      ires = icnt
      goto 20
    end if
  end do
  goto 10
20 if (.not.get_token(tline, token)) goto 10
  do icnt = 1, num_res
    if (getResData(template_handle, FrameIndex, icnt, ResRec))
continue
    if (ljust(ResRec.residue_number)) continue
    if (compstr(ResRec.residue_number, token)) then
      jres = icnt
      goto 30
    end if
  end do
  write (6,‘(‘‘ERROR: Unable to find residue ’’,a50)’) token
  goto 10
30 continue
c
  numTemplateRes = jres − ires + 1
  do icnt = 1, numTemplateRes
    TemplateResList(icnt) = ires + icnt−1
  end do
  if (numTemplateRes .eq. 1) then
    cys_idx = 1
  else
    write (6,‘(5x, ‘‘Enter relative position of cysteine : ’’,$)’)
    read(5,*) cys_idx
  end if
  write (6,‘(5x,‘‘Enter the RMS cutoff : ’’,$)’)
  read (5,*) RMS_cutoff
c
c...Collect template residue atoms for fitting (N/CA/C/O).
c
  numTemplateVec = 0
  do icnt = 1, numTemplateRes
    ires = TemplateResList(icnt)
    if (.not.getAtomOfRes(template_handle, FrameIndex, ires, ‘N’,
AtomRec)) then
      write (6,‘(‘‘ERROR: Unable to get N of template residue
’’,i4)’) ires
      call exit
    else
      numTemplateVec = numTemplateVec + 1
      TemplateVecArray(numTemplateVec) = AtomRec.vector
    end if
    if (.not.getAtomOfRes(template_handle, FrameIndex, ires, ‘CA’,
AtomRec)) then
      write (6,‘(‘‘ERROR: Unable to get CA of template residue
’’,i4)’) ires
      call exit
    else
      numTemplateVec = numTemplateVec + 1
      TemplateVecArray(numTemplateVec) = AtomRec.vector
    end if
    if (.not.getAtomOfRes(template_handle, FrameIndex, ires, ‘C’,
AtomRec)) then
      write (6,‘(‘‘ERROR: Unable to get C of template residue
’’,i4)’) ires
      call exit
    else
      numTemplateVec = numTemplateVec + 1
      TemplateVecArray(numTemplateVec) = AtomRec.vector
    end if
    if (.not.getAtomOfRes(template_handle, FrameIndex, ires, ‘O’,
AtomRec)) then
      write (6,‘(‘‘ERROR: Unable to get O of template residue
’’,i4)’) ires
      call exit
    else
      numTemplateVec = numTemplateVec + 1
      TemplateVecArray(numTemplateVec) = AtomRec.vector
    end if
  end do
c
c...Construct residue filter based on internal angles from the
template.
c
  if (.not. initializeResFilter(FragmentFilter, MAX_RMS_ATOMS))
then
    write(6, ‘(2X, ‘‘ERROR: Unable to make residue-filter
record’’)’)
    call exit
  end if
  FragmentFilter(1).seq_len = numTemplateRes
  FragmentFilter(1).start_residue = 2
  do icnt = 1, numTemplateRes
    ires = TemplateResList(icnt)
    if (.not.GetResData(template_handle, FrameIndex, ires,
ResRec)) then
      write (6,‘(‘‘ERROR: Unable to get record for residue
’’,i4)’) ires
      call exit
    end if
    FragmentFilter(icnt).phi_val = ResRec.phi_val
    FragmentFilter(icnt).phi_tol = angle_tol
    FragmentFilter(icnt).psi_val = ResRec.psi_val
    FragmentFilter(icnt).psi_tol = angle_tol
  end do
  FragmentFilter(cys_idx).residue_name = ‘CYS’
  if (returnTrajectory(template_handle)) continue
c
  call getenv (‘RSM_PDB_LISTFILE’, listfile)
  if (listfile.eq.‘ ’) then
    write (6,‘(/,‘‘Enter structure listfile : ’’,$)’)
    read (5,‘(a)’) listfile
  end if
  open (file=listfile, unit=LISTin, status=“old”)
c
  write (6,‘(/,‘‘Enter output logfile : ’’,$)’)
  read (5,‘(a)’) tline
  open (file=tline, unit=LOGout, status=“unknown”)
  write (6,‘(‘‘Enter output PDBfile : ’’,$)’)
  read (5,‘(a)’) tline
  open (file=tline, unit=PDBout, status=“unknown”)
c
c...Main loop
c
  50 read (LISTin, ‘(a)’, end=999) full_name
  if (full_name(1:1).eq.‘#’) goto 50
  if (parse_filename(full_name, file_path, file_name, file_root,
file_ext)) continue
  len_name = index(file_root, ‘ ’) − 1
c
  if (.not. readPDBFile(full_name, data_handle)) then
    write (6, ‘(2X, ‘‘**Unable to read PDB file’’)’)
    go to 100
  end if
c
c...Select only fragments containing cysteines.
c
  if (selectResByFilter(data_handle, FrameIndex, FragmentFilter,
numHitRes, HitResList)) continue
  if (numHitRes .eq. 0) goto 100
c
c...Get list of cysteines participating in disulfide bonds.
c
  call find_disulfide_pairs(data_handle, FrameIndex, MAX_RES,
numSS,
  . SS_1, SS_2)
  if (numSS .eq. 0) goto 100
c
c...Loop through fragments. Test whether: (a) cys_idx'th residue is
participating in a disulfide and
c (b) whether the fragment has an acceptable RMS overlap with the
template coordinates.
c
  do 90, icnt = 1, numHitRes
    icys = HitResList(icnt) + cys_idx − 1
    jcys = 0
    do jcnt = 1, numSS
      if (SS_1(jcnt).eq.icys) then
        jcys = SS_2(jcnt)
      else if (SS_2(jcnt).eq.icys) then
        jcys = SS_1(jcnt)
      end if
  end do
  if (jcys .eq. 0) goto 90
c
c...Extract coordinates for RMS test
c
  numFragAtom = 0
  do jcnt = 1, numTemplateRes
    jres = HitResList(icnt) + jcnt − 1
    if (.not.getAtomOfRes(data_handle, FrameIndex, jres, ‘N’,
AtomRec)) then
      write (6,‘(‘‘ERROR: Unable to get N of fragment residue
’’,i4)’) jres
      goto 90
    else
      numFragAtom = numFragAtom + 1
      FragAtomList(numFragAtom) = AtomRec.index
    end if
    if (.not.getAtomOfRes(data_handle, FrameIndex, jres, ‘CA’,
AtomRec)) then
      write (6,‘(‘‘ERROR: Unable to get CA of fragment residue
’’,i4)’) jres
      goto 90
    else
      numFragAtom = numFragAtom + 1
      FragAtomList(numFragAtom) = AtomRec.index
    end if
    if (.not.getAtomOfRes(data_handle, FrameIndex, jres, ‘C’,
AtomRec)) then
      write (6,‘(‘‘ERROR: Unable to get C of fragment residue
’’,i4)’) jres
      goto 90
    else
      numFragAtom = numFragAtom + 1
      FragAtomList(numFragAtom) = AtomRec.index
    end if
    if (.not.getAtomOfRes(data_handle, FrameIndex, jres, ‘O’,
AtomRec)) then
      write (6,‘(‘‘ERROR: Unable to get O of fragment residue
’’,i4)’) jres
      goto 90
    else
      numFragAtom = numFragAtom + 1
      FragAtomList(numFragAtom) = AtomRec.index
    end if
    do iatom = 1, numFragAtom
      jatom = FragAtomList(iatom)
      if (.not.getAtomData(data_handle, FrameIndex, jatom,
AtomRec)) then
        write (6,‘(‘‘ERROR: Unable to get record for fragment
atom’’,i6)’) jatom
        goto 90
      else
        FragmentVecArray(iatom) = AtomRec.vector
      end if
    end do
  end do
c
c...RMS Fit to template.
c
  call RMS_FIT(numTemplateVec, TemplateVecArray,
FragmentVecArray, RMS_WT, RMS_VALUE, t1, t2, r2)
  t2.x = −1.0 * t2.x
  t2.y = −1.0 * t2.y
  t2.z = −1.0 * t2.z
  if (RMS_VALUE .gt. RMS_cutoff) goto 90
c
c...Extract remaining atoms for fragment.
c
  if (.not.getAtomOfRes(data_handle, FrameIndex, icys, ‘CB’,
AtomRec)) then
    write (6,‘(‘‘ERROR: Unable to get CB of fragment residue
’’,i4)’) icys
    goto 90
  else
    numFragAtom = numFragAtom + 1
    FragAtomList(numFragAtom) = AtomRec.index
  end if
  if (.not.getAtomOfRes(data_handle, FrameIndex, icys, ‘SG’,
AtomRec)) then
    write (6,‘(‘‘ERROR: Unable to get CB of fragment residue
’’,i4)’) icys
    goto 90
  else
    numFragAtom = numFragAtom + 1
    FragAtomList(numFragAtom) = AtomRec.index
  end if
  if (.not.getAtomOfRes(data_handle, FrameIndex, jcys, ‘CA’,
AtomRec)) then
    write (6,‘(‘‘ERROR: Unable to get CA of fragment residue
’’,i4)’) jcys
    goto 90
  else
    numFragAtom = numFragAtom + 1
    FragAtomList(numFragAtom) = AtomRec.index
  end if
  if (.not.getAtomOfRes(data_handle, FrameIndex, jcys, ‘CB’,
AtomRec)) then
    write (6,‘(‘‘ERROR: Unable to get CB of fragment residue
’’,i4)’) jcys
    goto 90
  else
    numFragAtom = numFragAtom + 1
    FragAtomList(numFragAtom) = AtomRec.index
  end if
  if (.not.getAtomOfRes(data_handle, FrameIndex, jcys, ‘SG’,
AtomRec)) then
    write (6,‘(‘‘ERROR: Unable to get CB of fragment residue
’’,i4)’) jcys
      goto 90
    else
      numFragAtom = numFragAtom + 1
      FragAtomList(numFragAtom) = AtomRec.index
    end if
    call index_int_array(numFragAtom, FragAtomList, FragAtomIndex)
    call reorder_int_array(numFragAtom, FragAtomList,
FragAtomIndex)
c
c...Construct fragment object and apply transformations.
c
    if (getResData(data_handle, 1, icys, ResRec)) continue
    if (ResRec.ChainID.ne.‘ ’) then
      first_resnumber = ResRec.ChainID //
ResRec.residue_number(1:6)
    else
      first_resnumber = ResRec.residue_number(1:6)
    end if
    full_structure_name =
file_root(1:len_name)//‘_’//first_resnumber
c
    if (make_trj_from_atom_list(data_handle, INT_ONE, INT_ONE,
numFragAtom, FragAtomList,
      fragment_handle)) continue
    call rsm_translate_frame(fragment_handle, INT_ONE, t2)
    call rsm_rotate_frame(fragment_handle, INT_ONE, r2)
    call rsm_translate_frame(fragment_handle, INT_ONE, t1)
    call append_fragment(fragment_handle, full_structure_name,
PDBout, .FALSE.)
    write (LOGout, ‘(a22,1x,f5.2)’) full_structure_name, RMS_value
    if (returnTrajectory(fragment_handle)) continue
c
  90 end do
  100 if (returnTrajectory(data_handle)) continue
      goto 50
  999 close(LISTin)
      close(PDBout)
      close(LOGout)
      call exit
      end
```

### EXAMPLE 2

Cloning and Mutagenesis of Human IL-2

Interleukin-2 (IL-2) (accession number SWS P01585) is a cytokine with a predominant role in the proliferation of activated T helper lymphocytes. Mitogenic stimuli or interaction of the T cell receptor complex with antigen/MHC complexes on antigen presenting cells causes synthesis and secretion of IL-2 by the activated T cell, followed by clonal expansion of the antigen-specific cells. These effects are known as autocrine effects. In addition, IL-2 can have paracrine effects on the growth and activity of B cells and natural killer (NK) cells. These outcomes are initiated by interaction of IL-2 with its receptor on the T cell surface. Disruption of the IL-2/IL-2R interaction can suppress immune function, which has a number of clinical indications, including graft vs. host disease (GVHD), transplant rejection, and autoimmune disorders such as psoriasis, uveitis, rheumatoid arthritis, and multiple sclerosis. There is structural information available of the C125A mutant [3INK, Mc Kay, D. B. & Brandhuber, B. J., Science 257: 412 (1992)].

Cloning of Human IL-2

Numbering of the wild type and mutant IL-2 residues follows the convention of the first amino acid residue (A) of the mature protein being residue number 1 independent of any presequence e.g. met for the E. coli produced protein [see Taniguchi, T., et al., Nature 302: 305-310 (1983) and Devos, R., et al., Nucleic Acids Res. 11: 4307-4323 (1983)].

The DNA sequence encoding human Interleukin-2 (IL-2) was isolated from plasmid pTCGF-11 (ATCC). PCR primers were designed to contain restriction endonuclease sites NdeI and XhoI for subcloning into a pRSET expression vector (Invitrogen).

| | | |
|---|---|---|
| 1L2 Forward | GGAATTCCATATGGCACCTACTTCAAGTTCTACAAAGAAAACA | SEQ ID NO:1 |
| 1L2 Reverse | CCGCTCGAGTCAAGTTAGTGTTGAGATGATGCTTTGACA | SEQ ID NO:2 |

Double-stranded IL-2/pRSET was prepared by the following procedure. The PCR product containing the IL-2 sequence and pRSET were both cut with restriction endonucleases (1 μl PCR product, 1 μl each endonuclease, 2 μM appropriate 10×buffer, 15 μl water; incubated at 37° C. for 2 hours). The products of nuclease cleavage were isolated from an agarose gel (1% agarose, TAE buffer) and ligated together using T4 DNA ligase (80 ng IL-2 sequence, 160 ng pRSET vector, 4 μl 5×ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligase reaction mixture was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 5 ml 2YT media for 18 hours. Cells were then isolated and double-stranded DNA extracted from the cells using a Qiagen DNA miniprep kit.

Generation of IL-2 Cys Mutations

Site-directed mutants of IL-2 were prepared by the single-stranded DNA method (modification of Kunkel, T. A., Proc. Natl. Acad. Sci. U.S.A. 83: 488-492 (1985). Oligonucleotides were designed to contain the desired mutations and 15-20 bases of flanking sequence.

The single-stranded form of the IL-2/pRSET plasmid was prepared by transformation of double-stranded plasmid into the CJ236 cell line (1 μl IL-2/pRSET double-stranded DNA, 2 μl 2×KCM salts, 7 μl water, 10 μl PEG-DMSO competent CJ236 cells; incubated at 4° C. for 20 minutes and 25° C. for 10 minutes; plated on LB/agar with 100 μg/ml ampicillin and incubated at 37° C. overnight). Single colonies of CJ236 cells were then grown in 50 ml 2YT media to midlog phase; 5 μl VCS helper phage (Stratagene) were then added and the mixture incubated at 37° C. overnight. Single-stranded DNA was isolated from the supernatant by precipitation of phage (⅕ volume 20% PEG 8000/2.5 M NaCl; centrifuge at 12K for 15 minutes.). Single-stranded DNA was then isolated from phage using Qiagen single-stranded DNA kit. Sequencing identified a leucine-25 to serine mutation, which was corrected by mutagenesis using the “S25L” oligonucleotide.

S25L TAATTCCATTCAAAATCATCTGTA SEQ ID NO: 3

Mutagenic Oligonucleotides

| | | |
|---|---|---|
| N30C | GGTGAGTTTGGGATTCTTGTAACAATTAATTCCATTCAAAATCATCTG | SEQ ID NO:4 |
| Y31C | GGTGAGTTTGGGATTCTTACAATTATTAATTCCATTC | SEQ ID NO:5 |
| K32C | GGTGAGTTTGGGATTACAGTAATTATTAATTCC | SEQ ID NO:6 |
| N33C | CCTGGTGAGTTTGGCACACTTGTAATTATTAATTCC | SEQ ID NO:7 |
| K35C | GCATCCTGGTGAGACAGGGATTCTTGTAATTATTAATTCC | SEQ ID NO:8 |
| R38C | CTTAAATGTGAGCATACAGGTGAGTTTGGGATTC | SEQ ID NO:9 |
| F42C | GGGCATGTAAAACTTACATGTGAGCATCCTGG | SEQ ID NO:1O |
| K43C | CTTGGGCATGTAAAAACAAAATGTGAGCATCC | SEQ ID NO:11 |
| Y45C | GGCCTTCTTGGGCATACAAAACTTAAATGTGAGC | SEQ ID NO:12 |
| E68C | CTCAAACCTCTGGAGTGTGTGCTAAATTTAGC | SEQ ID NO:13 |
| L72C | GTTTTTGCTTTGAGCACAATTTAGCACTTCCTCC | SEQ ID NO:14 |
| N77C | CCTGGGTCTTAAGTGAAAACATTTGCTTTGAGCTAAATTTAGC | SEQ ID NO:15 |
| Y31C | GGGCATGTAAAAACAAAATGTGAGCATCCTGGTGAGTTTGGGATTCTTA | SEQ ID NO:16 |
| K43C | CAATTATTAATTCC | |

There was an additional double mutant made, L72C K43C, using the oligonucleotides corresponding to K43C and L72C single mutants (SEQ ID NO: 11 and SEQ ID NO: 14 respectively).

Site-directed mutagenesis was accomplished as follows: Mutagenesis oligonucleotides were dissolved to a concentration of 10 OD and phosphorylated on the 5′ end (2 μl oligonucleotide, 2 μl 10 mM ATP, 2 μl 10×Tris-magnesium chloride buffer, 1 μl 100 mM DTT, 10 μl water, 1 μl T4 PNK; incubate at 37° C. for 45 minutes.). Phosphorylated oligonucleotides were then annealed to single-stranded DNA template (2 μl single-stranded plasmid, 1 μl oligonucleotide, 1 μl 10×TM buffer, 6 μl water; heat at 94° C. for 2 minutes, 50° C. for 5 minutes, cool to room temperature). Double-stranded DNA was then prepared from the annealed oligonucleotide/template (add 2 μl 10×TM buffer, 2 μl 2.5 mM dNTPs, 1 μl 100 mM DTT, 1.5 μl 10 mM ATP, 4 μl water, 0.4 μl T7 DNA polymerase, 0.6 μl T4 DNA ligase; incubate at room temperature for 2 hours). E. coli (XL1 blue, Stratagene) was then transformed with the double-stranded DNA (1 μl double-stranded DNA, 10 μl 5×KCM, 40 μl water, 50 μl DMSO competent cells; incubate 20 minutes at 4° C., 10 minutes at room temperature), plated onto LB/agar containing 100 μg/ml ampicillin, and incubated at 37° C. overnight. Approximately four colonies from each plate were used to inoculate 5 ml 2YT containing 100 μg/ml ampicillin; these cultures were grown at 37° C. for 18-24 hours. Plasmids were then isolated from the cultures using Qiagen miniprep kit. These plasmids were sequenced to determine which IL-2/pRSET clones contained the desired mutation.

Sequencing Primers

| | | |
|---|---|---|
| Forward primer, | AATACGACTCACTATAC | SEQ ID NO:17 |
| “T7” | | |
| Reverse primer, | TAGTTATTGCTCAGCGGTGG | SEQ ID NO:18 |
| “RSET REV” | | |

Expression of IL-2 Mutants

Mutant proteins were expressed as follows: IL-2/pRSET clones containing the mutation were transformed into BL21 DE3 pLysS cells (Invitrogen) (1 μl double-stranded DNA, 2 μl 5×KCM, 7 μl water, 10 μl DMSO competent cells; incubate 20 minutes at 4° C., 10 minutes at room temperature), plated onto LB/agar containing 100 μg/ml ampicillin, and incubated at 37° C. overnight. 10 ml cultures in 10 ml 2YT with 100 μg/ml ampicillin were grown overnight from single colonies. 100 ml 2YT/ampicillin (100 μg/ml) was inoculated with these overnight cultures and incubated at 37° C. for 3 hours. This culture was then added to 1.5 L 2YT/ampicillin (100 μg/ml) and incubated until late-log phase (absorbance at 600 nm˜0.8), at which time IPTG was added to a final concentration of 1 mM. Cultures were incubated at 37° C. for another 3 hours and then cells were pelleted (10 Krpm, 10 minutes) and frozen at −20° C. overnight.

IL-2 mutants were then purified from the frozen cell pellets. First, cells were lysed in a microfluidizer (100 ml Tris EDTA buffer, 3 passes through a Microfluidizer [Microfluidics 110S]) and inclusion bodies were isolated by precipitation (10 Krpm, 10 minutes). Following cell lysis, 50 μl of cell material was saved for analysis by SDS-PAGE. All mutants expressed as determined by gel but several (e.g. E68C) precipitated on refolding. Inclusion bodies were then resuspended in 45 ml guanidine HCl and spun at 10 Krpm for 10 minutes. The supernatant was added to refolding buffer (45 ml guanidine HCl, 36 ml Tris pH 8, 231 mg cysteamine, 46 mg cystamine, 234 ml water) and incubated at room temperature for 3-5 hours. The mixture was then spun at 10 Krpm for 20 minutes, and the supernatant dialyzed 4-5 times in 5 volumes of buffer (10 mM ammonium acetate pH 6, 25 mM NaCl). The protein solution was then filtered through cellulose and injected onto an S Sepharose fast flow column (2.5 cm diameter×14 cm long) at 5 ml/min. The protein was then eluted using a gradient of 0-75% Buffer B over 60 minutes (Buffer A: 25 mM NH₄OAc, pH 6, 25 mM NaCl; Buffer B: 25 mM NH₄OAc, pH 6, 1 M NaCl). Purified protein was then exchanged into the appropriate buffer for the TETHER assay (typically 100 mM Hepes, pH 7.4). Average yields were 0.5 to 4 mg/L culture.

### EXAMPLE 3

Cloning and Mutagenesis of Human IL-4

IL-4 (accession number SWS P05112) is a cytokine that is critical for early immune response and allergic response; its interaction with the IL-4R is involved in the generation of Th2 cells. IL-4 recruits and activates B-cells that produce IgE (immunoglobulin E), eosinophils, and mast cells. These cells in turn tag and attack parasites in skin and in mucosal tissues and eject them from these tissues. The role of the IL-4/IL4R interaction in immune and allergic responses suggests that disruption of this interaction may alleviate such conditions as asthma, dermatitis, conjunctivitis, and rhinitis. There are crystal structures of IL-4 in isolation and in co-complex with a receptor molecule [1HIK, Muller, T. & Buehner, M., J Mol Biol 247: 360-372 (1995); with receptor alpha, 1IAR, Hage, T., et al., Cell 97: 271-281 (1999)].

Cloning of Human IL-4

Numbering of the wild type and mutant IL-4 residues follows the convention of the first amino acid residue (H) of the mature protein being residue number 1 independent of any presequence e.g. met for the E. coli produced protein [Yokota, T., et al., Proc. Natl. Acad. Sci. U.S.A. 83: 5894-5898 (1986)]. IL-4 lacking the secretion signal and containing an additional N-terminal methionine was expressed intracellularly in E. coli from the Sunesis RSET.IL4 plasmid.

The DNA sequence encoding human interleukin-4 (IL4) was isolated by PCR from the plasmid pcD-hIL-4 (ATCC Accession No. 57592) using PCR primers:

| | |
|---|---|
| 1L4 ForRse 5′ | |
| GGGTTTCATATGCACAAGTGCGATATCACCTT | SEQ ID NO:19 |
| 1L4 RevRse 5′ | |
| CCGCTCGAGTCAGCTCGAACACTTTGAATA | SEQ ID NO:20 |

These primers correspond to extracellular domain of the protein and which were designed to contain restriction endonuclease sites Nde I and XhoI for subcloning into a pRSET vector (Invitrogen). The PCR reaction was purified on a Qiaquick PCR purification column (Qiagen). The PCR product containing the IL4 sequence was cut with restriction endonucleases (41 μl PCR product, 2 μl each endonuclease, 5 μl appropriate 10×buffer; incubated at 37° C. for 90 minutes). The pRSET vector was cut with restriction endonucleases (6 μg DNA, 4 μl each endonuclease, 10 μl appropriate 10×buffer, water to 100 μl; incubated at 37° C. for 2 hours; add 2 μl CIP and incubated at 37° C. for 45 minutes). The products of nuclease cleavage were isolated from an agarose gel (1% agarose, TBE buffer) and ligated together using T4 DNA ligase (200 ng pRSET vector, 150 ng IL4 PCR product, 4 μl 5×ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit.

Generation of IL-4 Cysteine Mutations

Mutations were generated using as previously described [Kunkel, T. A., et al., Methods_{—}Enzymol. 154:367-82 (1987)]. DNA oligonucleotides used are shown below and were designed to hybridize with sense strand DNA from plasmid. Sequences were verified using primers with SEQ ID NO: 17 and SEQ ID NO: 18.

Mutagenic Oligonucleotides

| | | |
|---|---|---|
| Q8C | TTGATGATCTCACATAAGGTGA | SEQ ID NO:21 |
| E9C | AGTTTTGATGATACACTGTAAGGTGAT | SEQ ID NO:22 |
| K12C | GCTGTTCAAAGTGCAGATGATCTCCTG | SEQ ID NO:23 |
| S16C | CTGCTCTGTGAGGCAGTTCAAAGT | SEQ ID NO:24 |
| K37C | CAGTTGTGTTACAGGAGGCAGCAAAG | SEQ TD NO:25 |
| N38C | CCTTCTCAGTTGTGCACTTGGAGGC | SEQ ID NO:26 |
| K42C | GCAGAAGGTTTCACACTCAGTTGTG | SEQ ID NO:27 |
| Q54C | GGCTGTAGAAACACCGGAGCACAGTCG | SEQ ID NO:28 |
| Q78C | GAATCGGATCAGACACTTGTGCCTGTG | SEQ ID NO:29 |
| R81C | GCCGTTTCAGGAAGCAGATCAGCTGC | SEQ ID NO:30 |
| R85C | CCTGTCGAGACATTTCAGGAATCG | SEQ ID NO:31 |
| R88C | CCCAGAGGTTGCAGTCGAGCCG | SEQ ID NO:32 |
| N89C | CCCAGAGGCACCTGTCGAGCCG | SEQ ID NO:33 |
| N97C | CACAGGACAGGAACACAAGCCCGCC | SEQ ID NO:34 |
| K1O2C | CTGGTTGGCTTCACACACAGGACAGG | SEQ ID NO:35 |
| K117C | CTCTCATGATCGTGCATAGCCTTTCC | SEQ ID NO:36 |
| R121C | GAATATTTCTCACACATGATCGTC | SEQ ID NO:37 |

Expression of IL-4 Mutants

BL21 DE3 cells (Stratagene) were transformed with RSET.IL4 plasmids containing the described cysteine mutations and plated onto LB agar containing 100 μg/ml ampicillin. After overnight growth fresh individual colonies were used to inoculate a 37° C. overnight shake flask culture with 30 ml 2YT (with 50 μg/ml ampicillin) media. In the morning this overnight culture was used to inoculate 1.5 L of 2YT/ampicillin (50 μg/ml), which was further cultured at 37° C. and 200 rpm in a 4.0 L dented bottom shake flask. When the optical density of the culture at λ=600 reached 0.8 it was induced to produce IL-4 protein by the addition of 1 mM IPTG. After 4 more hours of incubation the cultures were harvested, the cells pelleted by centrifugation at 7K rpm for 10 minutes (K-9 Komposite Rotor), and frozen at −20° C.

The cell pellet was then thawed and resuspended in 100 ml of 10 mM Tris pH 8, 50 mM NaCl and 1 mM EDTA. This solution was kept chilled and run through a microfluidizer twice (model 110S Microfluidics Corp, Newton Mass.), and centrifuged at 7K rpm for 15 minutes). The pellet containing the IL-4 inclusion bodies was then resuspended in a 50 ml solution of 5 M guanidine HCl, 50 mM Tris pH 8, 50 mM NaCl, 2.5 mM reduced glutathione, and 0.25 mM oxidized glutathione, and incubated for one hour at room temperature with gentle mixing. The solubilized protein solution was then centrifuged at 7.5K rpm for 15 minutes and the supernatant 0.45 μm filtered to remove insoluble debris.

The IL-4 was refolded by slowly adding the filtered solution to 9 volumes (450 ml) of 50 mM Tris pH 8, 50 mM NaCl, 2.5 mM reduced glutathione and 0.25 mM oxidized glutathione over a 30 minute period. The resulting solution was further incubated with slow stirring for 3 hours at room temperature, then placed in a 3000 mwco dialysis bag and exchanged 3 times against 20 L of 0.5×PBS (phosphate-buffered saline).

The refolded mutant proteins were then purified using a Hi-S Column Cartridge (Bio-Rad). After clarifying the protein solution by centrifugation and filtration it was loaded onto the column at a 5 ml/min flow rate. The column was next washed with buffer A (0.5×PBS) for 15-20 minutes, and 1.5 minute 7.5 ml fractions were collected over a 0-100% gradient between Buffer A and Buffer B (PBS, 1M NaCl). The fractions that contained the IL-4 protein as determined by SDS-PAGE and optical density as 280 nm were pooled, concentrated with a 5K mwco filter, and their buffer exchanged to PBS. This solution was then 0.2 μm filtered, frozen in ethanol dry ice bath, and stored at −80° C.

### EXAMPLE 4

### Cloning and Mutagenesis of Human Tumor Necrosis Factor—Alpha (TNF-α)

Tumor necrosis factor-α (TNF-α) (accession number SWS P01375) is a cytokine produced mainly by activated macrophages, and it plays a critical role in immune responses including septic shock, inflammation, and cachexia. This protein can interact with two receptors, TNF R1 and TNF R2. These two receptors share no similarity in their intracellular domains, which suggests that they are involved in different signal transduction pathways. A structure of TNF-α is available [1TNF, Eck, M. J., et al., J Biol Chem 264: 17595-17605(1989)]; TNF-α is an elongated beta sheet, and it forms a trimer. Mutation of some of the intersubunit residues of the trimer indicates that they form part of the binding site to the receptor. However, there is no structure of TNF bound to a receptor to date.

Cloning of human TNF-α

The DNA sequence encoding human Tumor Necrosis Factor (TNF) was isolated by PCR from the plasmid pUC-RI-4large (ATCC #65947) using PCR primers listed below corresponding to extracellular domain of the protein and which were designed to contain restriction endonuclease sites Nde I and XhoI for subcloning into a pRSET vector (Invitrogen).

| | |
|---|---|
| TNF RSET For 5′ | |
| GGGTTTCATATGGTCCGTTCATCTTCTCGAAC | SEQ ID NO:38 |
| TNF RSET Rev 5′ | |
| CCGCTCGAGTCACAGGGCAATGATCCCAA | SEQ ID NO:39 |

The PCR reaction was purified on a Qiaquick PCR purification column (Qiagen). The PCR product containing the TNF sequence was cut with restriction endonucleases (41 μl PCR product, 2 μl each endonuclease, 5 μl appropriate 10×buffer; incubated at 37° C. for 90 minutes). The pRSET vector was cut with restriction endonucleases (6 μg DNA, 4 μl each endonuclease, 10 μl appropriate 10×buffer, water to 100 μl; incubated at 37° C. for 2 hours; added 2 μl CIP and incubated at 37° C. for 45 minutes). The products of nuclease cleavage were isolated from an agarose gel (1% agarose, TBE buffer) and ligated together using T4 DNA ligase (200 ng pRSET vector, 150 ng TNF PCR product, 4 μl 5×ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit. Sequencing of TNF genes was accomplished using primers having SEQ ID NO: 17 and SEQ ID NO: 18.

Generation of TNF-α Cysteine Mutations

Mutations were generated using as previously described [Kunkel, T. A., et al., Methods_{—}Enzymol. 154: 367-82 (1987)]. DNA oligonucleotides used are shown below and were designed to hybridize with sense strand DNA from plasmid. Sequences of the mutants were verified using primers with SEQ ID NO: 17 and SEQ ID NO: 18.

Mutagenic Oligonucleotides

| | |
|---|---|
| R32C | |
| GAGGGCATTGGCGCAGCGGTTCAGCCAC | SEQ ID NO:40 |
| A33C | |
| CAGGAGGGCATTGCACCGGCGGTTCAG | SEQ ID NO:41 |
| N34C | |
| GGCCAGGAGGGCACAGGCCCGGCGGTTC | SEQ ID NO:42 |
| R44C | |
| CAGCTGGTTATCACACAGCTCCACGCC | SEQ ID NO:43 |
| Q47C | |
| TGGCACCACCAGGCAGTTATCTCTCAG | SEQ ID NO:44 |
| T72C | |
| GAGGAGCACATGGCAGGAGGGGCAGCC | SEQ ID NO:45 |
| H73C | |
| GGTGAGGAGCACACAGGTGGAGGGGCAG | SEQ ID NO:46 |
| L75C | |
| GGTGTGGGTGAGGCACACATGGGTGGAG | SEQ ID NO:47 |
| T77C | |
| GCTGATGGTGTGGCAGAGGAGCACATG | SEQ ID NO:48 |
| V91C | |
| CAGAGAGGAGGTTGCACTTGGTCTGGTAG | SEQ ID NO:49 |
| N92C | |
| GGCAGAGAGGAGGCAGACCTTGGTCTG | SEQ ID NO:50 |
| 595C | |
| GCTCTTGATGGCACAGAGGAGGTTGAC | SEQ ID NO:51 |
| E104C | |
| CCTCAGCCCCCTCTGGGGTGCACCTCTGGCAGGGG | SEQ ID NO:52 |
| T105C | |
| CCTCAGCCCCCTCTGGGCACTCCCTCTGGCAGGGG | SEQ ID NO:53 |
| E107C | |
| GGCCTCAGCCCCGCATGGCGTCTCCCTCTGGC | SEQ ID NO:54 |
| E110C | |
| CCAGGGCTTGGCGCAAGCCCCCTCTGGGG | SEQ ID NO:55 |
| A111C | |
| ATACCAGGGCTTGCACTCAGCCCCCTC | SEQ ID NO:56 |
| K112C | |
| GGGTAGTTTCTGGCAAAATATGGCTTG | SEQ ID NO:57 |
| Q125C | |
| CACCCTTCTCCAGGCAGAAGACCCCTCC | SEQ ID NO:58 |
| R138C | |
| GCTGAGATCAATTGTCCCGACTATCTC | SEQ ID NO:59 |
| E146C | |
| GACCTGCCCAGAGCAGGCAAAGTCGAG | SEQ ID NO:60 |
| 5147C | |
| GTAGACCTGCCCACACTCGGCAAAGTC | SEQ ID NO:61 |

Expression of TNF-α Mutant Proteins

BL21 DE3 cells (Stratagene) were transformed with RSET TNF-α plasmids containing the described cysteine mutations and plated onto LB agar containing 100 μg/ml ampicillin. After overnight growth fresh individual colonies were used to inoculate a 37° C. overnight shake flask culture with 30 ml 2YT (with 50 μg/ml ampicillin) media. In the morning this overnight culture was used to inoculate 1.5 L of 2YT/ampicillin (50 μg/ml), which was further cultured at 37° C. and 200 rpm in a 4.0 L dented bottom shake flask. When the optical density of the culture at λ=550 reached 0.8 it was induced to produce TNF-α protein by the addition of 1 mM IPTG. After 4 more hours of incubation the cultures were harvested, the cells pelleted by centrifugation at 7K rpm for 10 minutes (K-9 Komposite Rotor), and frozen at −20° C.

The cell pellet was then thawed and resuspended in 100 ml of 25 mM ammonium acetate pH 6, 1 mM DTT and 1 mM EDTA. This solution was kept chilled and run through a microfluidizer twice (model 110S Microfluidics Corp, Newton Mass.), centrifuged at 9K rpm for 15 minutes to remove insoluble material and further clarified by 0.45 μm filtration. This solution was then loaded onto an S-Sepharose ff Column (Bio-Rad) column at a 5 ml/min flow rate. The flow rate was then increased to 7.5 mL/min for the following steps. The column was next washed with Buffer A (0.2 M ammonium acetate pH 6, 1 mM DTT) until the OD₂₈₀ approached zero (15-20 minutes), and fractions were collected over a 0-100% gradient in 60 minutes between Buffer A and Buffer B (1 M ammonium acetate pH 6, 1 mM DTT). The fractions that contained the TNF-α protein as determined by SDS-PAGE and optical density at 280 nm were pooled and placed in a 3000 mwco dialysis bag and dialyzed overnight at 4° C. against 4 L of 10 mM Tris pH 7.5, 10 mM NaCl, and 1 mM DTT. The dialyzed protein solution was then clarified by centrifuging at 13.5K rpm for 10 minutes filtering through a 0.2 μm filter.

The mutant proteins were then loaded onto a Q-Sepharose Column (Bio-Rad) at a 5 ml/min flow rate. The flow rate was increased to 7.5 mL/min for the following steps. The column was next washed with Buffer A (10 mM Tris pH 7.5, 10 mM NaCl, 1 mM DTT) until the OD₂₈₀ approached zero (15-20 minutes), and fractions were collected over a 0-100% gradient in 40 minutes between Buffer A and Buffer B (10 mM Tris pH 7.5, 0.5 M NaCl, 1 mM DTT). The fractions that contained the TNF-α protein as determined by SDS-PAGE and optical density at 280 nm were pooled and concentrated with a 5K mwco filter, and their buffer exchanged to PBS. This solution was then 0.2 μm filtered, frozen in ethanol dry ice bath, and stored at −80° C.

### EXAMPLE 5

### Cloning and Mutagenesis of Human Interleukin-1 Receptor Type I (IL-1RI)

Binding of the IL-1 receptor (accession number SWS P14778) to IL-1alpha or IL-1beta is another important mediator of immune and inflammatory responses. This interaction is controlled by at least three mechanisms. Firstly, the protein IL-R2 binds to IL-1alpha and IL-1beta but does not signal. Secondly, proteolytically processed IL-1R1 and IL-1R2 are soluble and bind to IL-1 in circulation. Finally there exists a natural IL-1R antagonist called IL-1ra, that functions by binding IL-1R1 and thereby blocking IL-1R1 binding of IL-1alpha and IL-1beta. Inhibition of these interactions with an orally available small molecule would be desirable in treatment of diseases such as rheumatoid arthritis, autoimmune disorders, and ischemia. Two structures of IL-1R have been solved [with a antagonist peptide, 1G0Y, Vigers, G. P. A., et al., J. Biol. Chem. 275:36927-36933 (2000); with receptor antagonist, 1IRA, Schreuder, H., et al., Nature 386: 194-200 (1997)].

Cloning of human IL-1 Receptor Type I

The IL-1 receptor has three regions: an N-terminal extracellular region, a transmembrane region, and a C-terminal cytoplasmic region. The extracellular region itself contains three immunoglobin-like C2-type domains. The constructs used here contain the two N-terminal domains of the extracellular region. Numbering of the wild type and mutant IL1R residues follows the convention of the first amino acid residue (L) of the mature protein being residue number 1 after processing of the signal sequence [Sims, J. E., et al., Proc. Natl. Acad. Sci. U.S.A. 86: 8946-8950 (1989)]. The sequence of the 2 domain protein is shown below as SEQ ID NO: 62.

| | |
|---|---|
| 1 | LEADKCKERE EKIILVSSAN EIDVRPCPLN PNEHKGTITW YKDDSKTPVS TEQASRIHQH |
| 61 | KEKLWFVPAK VEDSGHYYCV VRNSSYCLRI KISAKFVENE PNLCYNAQAI FKQKLPVAGD |
| 121 | GGLVCPYMEF FKNENNELPK LQWYKDCKPL LLDNIHFSGV KDRLIVMNVA EKHRGNYTCH |
| 181 | ASYTYLGKQY PITRVIEFIT LEENK |

In brief, cysteine mutants were made in the context of a 2 domain receptor and a 2 domain receptor with a his tag. In addition, the constructs possessed a mutation at a glycosylation site, and one construct possessed a mutation at a glycosylation site in addition to a deletion at the C-terminal residue of the 2 domain region. The assembly of these constructs is described below.

The DNA sequence encoding human Interleukin-1 receptor (IL1R) was isolated by PCR from a HepG2 cDNA library (ATCC) using PCR primers (IL1RsigintFor 5′; IL1RintRev 5′) corresponding to the signal sequence and the end of the extracellular domain of the protein.

| | |
|---|---|
| IL1RsigintFor | |
| TTACTCAGACTTATTTGTTTCATAGCTCTA | SEQ ID NO:63 |
| IL1RintRev | |
| GAAATTAGTGACTGGATATATTAACTGGAT | SEQ ID NO:64 |

The appropriate sized band was isolated from an agarose gel and used as the template for a second round of PCR using oligos (IL1RsigFor; IL1R319Rev), which were designed to contain restriction endonuclease sites EcoR and XhoI for subcloning into a pFBHT vector.

| | | |
|---|---|---|
| IL1Rsig For | CCGGAATTCATGAAAGTGTTACTCAGACTTATTTGTTTC | SEQ ID NO:65 |
| IL1R319 Rev | CCGCTCGAGTCACTTCTGGAAATTAGTGACTGGATATATTAA | SEQ ID NO:66 |

The pFBHT vector is modified from the original pFastBac1(Gibco/BRL) by cloning the sequence for TEV protease followed by (His)₆ tag and a stop signal into the XhoI and HinDIII sites. The PCR product containing the IL1R sequence was cut with restriction endonucleases (41 μl PCR product, 2 μl each endonuclease, 5 μl appropriate 10×buffer; incubated at 37° C. for 90 minutes). The pFBHT vector was cut with restriction endonucleases (6 μg DNA, 4 μl each endonuclease, 10 μl appropriate 10×buffer, water to 100 μl; incubated at 37° C. for 2 hours; add 2 μl CIP and incubated at 37° C. for 45 minutes). The products of nuclease cleavage were isolated from an agarose gel (1% agarose, TBE buffer) and ligated together using T4 DNA ligase (200 ng pFBHT vector, 150 ng IL1R PCR product, 4 μl 5×ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit.

A 2-domain version of IL1R was created by PCR using the 3-domain IL1R-FBHT clone as a template. PCR was performed using the primers IL1RsigFor (SEQ ID NO: 65) corresponding to the signal sequence, in addition to one of the following two reverse primers. The reverse primers are IL1R2Drevstop-Xho, which corresponds to the end of the second extracellular domain of the protein with a stop signal, and IL1R2Drev-Xho, which corresponds to the end of the second extracellular domain of the protein without a stop signal to create a fusion with the TEV protease site and the His tag.

| | | |
|---|---|---|
| IL1R2Drevstop-Xho | CCGCTCGAGTCATCATTTGTTTTCCTCTAGAGTAATAAA | SEQ ID NO:67 |
| ILlR2Drev-Xho | CCGCTCGAGTCATTTGTTTTCCTCTAGAGTAATAAA | SEQ ID NO:68 |

The PCR primers contain restrictions sites (EcoRI at the 5′ end and XhoI at the 3′ end), which were used to ligate the 2-domain version into the pFBHT vector. The PCR product containing the IL1R2D sequence was cut with restriction endonucleases (41 μl PCR product, 2 μl each endonuclease, 5 μl appropriate 10×buffer; incubated at 37° C. for 90 minutes). The products of nuclease cleavage were isolated from an agarose gel (1% agarose, TBE buffer) and ligated together using T4 DNA ligase (200 ng pFBHT vector, 150 ng IL1R2D PCR product, 4 μl 5×ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit.

Additionally, the two glycosylation sites within IL1R2D, N83 and N176, were each individually mutated to a histidine, in order to make a more homogeneous protein. Each of these single mutants were made in the context of the 2-domain protein without a his tag (sIL1Rd2-FB) and the 2-domain protein with a his tag (sIL1Rd2-FBHT). Mutation was accomplished by PCR using two sets of primers to make two fragments, followed by stitching together of the fragments using the outside primers IL1RsigFor (SEQ ID NO: 65) and either IL1R2Drevstop-Xho (SEQ ID NO: 67) or IL1R2Drev-Xho (SEQ ID NO: 68) as described below. Brief descriptions of the 2-domain glycosylation mutants and their construction follow.

The construct for the N83H mutant without a his tag is referred to as sIL1R2D-N83H-FB, and it was created using IL1RsigFor (SEQ ID NO: 65) and N83HR (SEQ ID NO: 69) along with N83HF (SEQ ID NO: 70), and IL1R2Drevstop-Xho (SEQ ID NO: 67)

| | | |
|---|---|---|
| N83HR | GAGGCAGTAAGATGAATGTCTTACC | SEQ ID NO:69 |
| N83HF | CTATTGCGTGGTAAGACATTCATCTT | SEQ ID NO:70 |

The construct for the N83H mutant with a his tag is referred to as sIL1R2D-N83H-FBHT and was created using IL1RsigFor (SEQ ID NO: 65), and N83HR (SEQ ID NO: 69) along with N83HF (SEQ ID NO: 70) and IL1R2Drev-Xho (SEQ ID NO: 68).

The construct for the N176H mutant without a his tag is referred to as sIL1R2D-N176H-FB and it was created using IL1RsigFor (SEQ ID NO: 65), N176HR (SEQ ID NO: 71), N176HF (SEQ ID NO: 72), and IL1R2Drevstop-Xho (SEQ ID NO: 67).

| | |
|---|---|
| NI76HR | |
| ATGACAAGTATAGTGCCCTCTATGCTTTTCACG | SEQ ID NO:71 |
| N176HF | |
| GCTGAAAAGCATAGAGGGCACTATACTTGTCAT | SEQ ID NO:72 |

The construct for the N176H mutant with a his tag is referred to as sIL1R2D-N176H-FBHT.and it was created using IL1RsigFor (SEQ ID NO: 65), and N176HR (SEQ ID NO: 71), along with N176HF (SEQ ID NO: 72), and IL1R2Drev-Xho (SEQ ID NO: 68).

The PCR products were isolated from and agarose gel and PCR was used to sew the two fragments together using the IL1RsigFor (SEQ ID NO: 65) and IL1R2Drevstop-Xho (SEQ ID NO: 67) or IL1R2Drev-Xho primers (SEQ ID NO: 68). The PCR products containing the IL1R2D sequences mutated at the glycosylation site were cut with restriction endonucleases (41 μl PCR product, 2 μl each endonuclease, 5 μl appropriate 10×buffer; incubated at 37° C. for 90 minutes). The products of nuclease cleavage were isolated from an agarose gel (1% agarose, TBE buffer) and ligated together using T4 DNA ligase (200 ng PFBHT vector, 150 ng IL1R2D PCR product, 4 μl 5×ligase buffer [300 mM Tris pH 7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4° C. for 20 minutes, 25° C. for 10 minutes), and plated onto LB/agar plates containing 100 μg/ml ampicillin. After incubation at 37° C. overnight, single colonies were grown in 3 ml 2YT media for 18 hours. Cells were then isolated and double-stranded DNA extracted from the cells using a Qiagen DNA miniprep kit. The subsequent plasmids are referred to as sIL1R2D-N83H-FB or sIL1R2D-N83H-FBHT and as sIL1R2D-N176H-FB or as sIL1R2D-N176H-FBHT.

Finally, an additional construct was made using the sIL1R2D-N83H-FB construct. The additional construct contains the 2-domain IL1R receptor without a his tag and with two mutations: a N83H glycosylation mutation and a deletion of the C-terminal residue (K205). This construct is named sIL1R2D2M-FB, and was made using the K205del oligonucleotide.

K205del CTCGAGTCATCAGTTTTCCTCTAG SEQ ID NO: 73

Generation of IL-1RI Cysteine Mutations

Site-directed mutants of IL1R2D were prepared by the single-stranded DNA method [modification of Kunkel, T. A., Proc. Natl. Acad. Sci. U.S.A. 82: 488-492 (1985)]. Oligonucleotides were designed to contain the desired mutations and 15-20 bases of flanking sequence.

The single-stranded form of the IL1R2D (sIL1R2D-FBHT, sIL1R2D-N176H-FB/FBHT, sIL1R2D-N83H-FB/FBHT, sIL1R2D2M-FB) plasmid was prepared by transformation of double-stranded plasmid into the CJ236 cell line (1 μl IL1R-FB double-stranded DNA, 2 μl 2×KCM salts, 7 μl water, 10 μl PEG-DMSO competent CJ236 cells; incubated at 4° C. for 20 minutes and 25° C. for 10 minutes; plated on LB/agar with 100 μg/ml ampicillin and incubated at 37° C. overnight). Single colonies of CJ236 cells were then grown in 50 ml 2YT media to midlog phase; 10 μl VCS helper phage (Stratagene) were then added and the mixture incubated at 37° C. overnight. Single-stranded DNA was isolated from the supernatant by precipitation of phage (⅕ volume 20% PEG 8000/2.5 M NaCl; centrifuge at 12K for 15 minutes.). Single-stranded DNA was then isolated from phage using Qiagen single-stranded DNA kit.

Site-directed mutagenesis was accomplished as follows. Oligonucleotides were dissolved to a concentration of 10 OD and phosphorylated on the 5′ end (2 μl oligonucleotide, 2 μl 10 mM ATP, 2 μl 10×Tris-magnesium chloride buffer, 1 μl 100 mM DTT, 10 μl water, 1 μl T4 PNK; incubate at 37° C. for 45 minutes). Phosphorylated oligonucleotides were then annealed to single-stranded DNA template (2 μl single-stranded plasmid, 1 μl oligonucleotide, 1 μl 10×TM buffer, 6 μl water; heat at 94° C. for 2 minutes, 50° C. for 5 minutes, cool to room temperature). Double-stranded DNA was then prepared from the annealed oligonucleotide/template (add 2 μl 10×TM buffer, 2 μl 2.5 mM dNTPs, 1 μl 100 mM DTT, 1.5 μl 10 mM ATP, 4 μl water, 0.4 μl T7 DNA polymerase, 0.6 μl T4 DNA ligase; incubate at room temperature for two hours). E. coli (XL1 blue, Stratagene) was then transformed with the double-stranded DNA (1 μl double-stranded DNA, 10 μl 5×KCM, 40 μl water, 50 μl DMSO competent cells; incubate 20 minutes at 4° C., 10 minutes at room temperature), plated onto LB/agar containing 100 μg/ml ampicillin, and incubated at 37° C. overnight. Approximately four colonies from each plate were used to inoculate 5 ml 2YT containing 100 μg/ml ampicillin; these cultures were grown at 37° C. for 18-24 hours. Plasmids were then isolated from the cultures using Qiagen miniprep kit. These plasmids were sequenced to determine which IL1R2D-FB clones contained the desired mutation.

Sequencing of IL1R2D genes was accomplished as follows. The concentration of plasmid DNA was quantitated by absorbance at 280 nm. 800 ng of plasmid was mixed with sequencing reagents (8 μl DNA, 3 μl water, 1 μl sequencing primer, 8 μl sequencing mixture with Big Dye [Applied Biosystems]). The sequencing primers used were FB Forward and FB Reverse, shown below.

| | | |
|---|---|---|
| FB Forward | TATTCCGGATTATTCATACC | SEQ ID NO:74 |
| FB Reverse | CCTCTACAAATGTGGTATGGC | SEQ ID NO:75 |

The mixture was then run through a PCR cycle (96° C., 10 s; 50° C., 5 s; 60° C. 4 minutes; 25 cycles) and the DNA reaction products were precipitated (20 μl mixture, 80 μl 75% isopropanol; incubated 20 minutes at room temperature, pelleted at 14 K rpm for 20 minutes; wash with 250 μl 70% ethanol; heat 1 minute at 94° C.). The precipitated products were then suspended in Template Suppression Buffer (TSB, Applied Biosystems) and the sequence read and analyzed by an Applied Biosystems 310 capillary gel sequencer. In general, 3 out of 4 of the plasmids contained the desired mutation. A listing of the constructs and their mutant(s) is given below, although any cysteine mutants can be made in any of the given contexts.

| Construct | Mutant(s) |
|---|---|
| sIL1R2D-N83H-FB | E11C, I13C, V16C, Q108C, I110C, K112C, |
| | K114C, V117C, V124C, Y127C, E129C |
| sIL1R2D-N83H-FBHT | E11C, I13C, V16C, Q108C, I110C, K112C, |
| | Q113C, K114C, V117C, V124C, Y127C, |
| | E129C |
| sIL1R2D-N176H-FB | E11C |
| sIL1R2D-N176H-FBHT | E11C, V16C, V124C, E129C |
| sIL1R2D2M-FB | E11C, K12C, I13C, A107C, K112C, V124C, |
| | Y127. |

Mutagenic Oligonucleotides

| | | |
|---|---|---|
| E11C | TAAAATTATTTTACATTCACGTTCC | SEQ ID NO:76 |
| Kl2C | CACTAAAATTATACATTCTTCACGTTC | SEQ ID NO:77 |
| 113C | TGACACTAAAATACATTTTTCTTCACG | SEQ ID NO:78 |
| V16C | ATTTGCAGATGAACATAAAATTATTT | SEQ ID NO:79 |
| A107C | AAATATGGCTTGGCAATTATAACATAAG | SEQ ID NO:80 |
| Q108C | CTTAAATATGGCGCATGCATTATAACA | SEQ ID NO:81 |
| I110C | GTTTCTGCTTAAAGCAGGCTTGTGCATT | SEQ ID NO:82 |
| K112C | GGGTAGTTTCTGACAAAATATGGC | SEQ ID NO:83 |
| Q113C | AACGGGTAGTTTACACTTAAATATGGC | SEQ ID NO:84 |
| K114C | CTGCAACGGGTACGCACTGCTTAAATATG | SEQ ID NO:85 |
| V117C | CTCCGTCTCCTGCACAGGGTAGTTTCTG | SEQ ID NO:86 |
| V124C | CATATAAGGGCAACAAGTCCTCC | SEQ ID NO:87 |
| Y127C | AAAAAACTCCATACAAGGGCACACAAG | SEQ ID NO:88 |
| E129C | TTTAAAAAAACACATATAAGGGCA | SEQ ID NO:89 |

Expression of IL-1 R Mutant Proteins

All IL1R-FB/FBHT plasmids were site-specifically transposed into the baculovirus shuttle vector (bacmid) by transforming the plasmids into DH10bac (Gibco/BRL) competent cells as follows: 1 μl DNA at 5 ng/μl, 10 μl 5×KCM [0.5 M KCl, 0.15 M CaCl₂, 0.25 M MgCl₂], 30 μl water was mixed with 50 μl PEG-DMSO competent cells, incubated at 4° C. for 20 minutes, 25° C. for 10 minutes, add 900 μl SOC and incubate at 37° C. with shaking for 4 hours, then plated onto LB/agar plates containing 50 μg/ml kanamycin, 7 μg/ml gentamycin, 10 μg/ml tetracycline, 100 μg/ml Bluo-gal, 10 μg/ml IPTG. After incubation at 37° C. for 24 hours, large white colonies were picked and grown in 3 ml 2YT media overnight. Cells were then isolated and double-stranded DNA was extracted from the cells as follows: pellet was resuspended in 250 μl of Solution 1 [15 mM Tris-HCl (pH 8.0), 10 mM EDTA, 100 μg/ml RNase A]. 250 μl of Solution 2 [0.2 N NaOH, 1% SDS] was added, mixed gently and incubated at room temperature for 5 minutes. 250 μl 3 M potassium acetate was added and mixed, and the tube placed on ice for 10 minutes. The mixture was centrifuged 10 minutes at 14,000×g and the supernatant transferred to a tube containing 0.8 ml isopropanol. The contents of the tube were mixed and placed on ice for 10 minutes; centrifuged 15 minutes at 14,000×g. The pellet was washed with 70% ethanol and air-dried and the DNA resuspended in 40 μl TE.

The bacmid DNA was used to transfect Sf9 cells. Sf9 cells were seeded at 9×10⁵ cells per 35 mm well in 2 ml of Sf-900 II SFM medium containing 0.5×concentration of antibiotic-antimycotic and allowed to attach at 27° C. for 1 hour. During this time, 5 μl of bacmid DNA was diluted into 100 μl of medium without antibiotics, 6 μl of CellFECTIN reagent was diluted into 100 μl of medium without antibiotics and then the 2 solutions were mixed gently and allowed to incubate for 30 minutes at room temperature. The cells were washed once with medium without antibiotics, the medium was aspirated and then 0.8 ml of medium was added to the lipid-DNA complex and overlaid onto the cells. The cells were incubated for 5 hours at 27° C., the transfection medium was removed and 2 ml of medium with antibiotics was added. The cells were incubated for 72 hours at 27° C. and the virus was harvested from the cell culture medium.

The virus was amplified by adding 0.5 ml of virus to a 50 ml culture of Sf9 cells at 2×10⁶ cells/ml and incubating at 27° C. for 72 hours. The virus was harvested from the cell culture medium and this stock was used to express the various IL1R constructs in High-Five cells. A 1 L culture of High-Five cells at 1×10⁶ cells/ml was infected with virus at an approximate MOI of 2 and incubated for 72 hours. Cells were pelleted by centrifugation and the supernatant was loaded onto an IL1R antagonist column at 1 ml/min, washed with PBS followed by a wash with Buffer A (0.2 M NaOAc pH 5.0, 0.2 M NaCl). The protein was eluted from the column by running a gradient from 0-100% of Buffer B (0.2 M NaOAc pH 2.5, 0.2 M NaCl) in 10 minutes followed by 15 minutes of 100% Buffer B at 1 ml/min collecting 2 ml fractions in tubes containing 300 μl of unbuffered Tris. The appropriate fractions were pooled, concentrated and dialyzed against 5 L of 50 mM Tris pH 8.0, 100 mM NaCl at 4° C. and filtered through a 0.2 μm filter.

### EXAMPLE 6

Cloning and Mutagenesis of Human Caspase-3 (CASP-3)

Caspase-3 (accession number SWS P42574) is one of a series of caspases involved in the apoptosis of cells. It exists as the inactive proform, and can be processed by caspases 8, 9, or 10 to form a small subunit and a large subunit, which heterodimerize to constitute the active form. Caspases that are substrates for caspase-3 in the cascade are caspase-6, caspase-7 and caspase-9. Caspase-3 has been shown to be the important for the cleavage of amyloid-beta precursor protein 4A. This cleavage has been linked to the deposition of Abeta peptide deposition and death of neurons in Alzheimers disease and hippocampal neurons following ischemic and exitoxic brain injury. There is a crystal structure available for caspase-3 [1CP3, Mittl, P. R., et al., J Biol Chem 272:6539-6547 (1997)].

Cloning of Human Caspase-3

The human version of caspase-3 (also known as Yama, CPP32 beta) was cloned directly from Jurkat cells (Clone E6-1; ATCC). Briefly, total RNA was purified from Jurkat cells growing at 37° C./5% CO₂ using Tri-Reagent (Sigma). Oligonucleotide primers were designed to allow DNA encoding the large and small subunits of Caspase-3/Yama/CPP32 to be amplified by polymerase chain reaction (PCR). Briefly, DNA encoding amino acids 28-175 (encompassing most of the large subunit) was directly amplified from 1 μg total RNA using Ready-To-Go-PCR Beads (Amersham/Pharmacia) and the following oligonucleotides:

| | |
|---|---|
| casp-3 large for | |
| TTCCATATGTCTGGAATATCCCTGGACAACAGTTA | SEQ ID NO:90 |
| casp-3 large rev | |
| AAGGAATTCTTAGTCTGTCTCAATGCCACAGTCCAG | SEQ ID NO:91 |

DNA encoding amino acids 176-277 (encompassing most of the small subunit) was directly amplified from 1 μg total RNA using Ready-To-Go-PCR Beads (Amersham/Pharmacia) and the following oligonucleotides:

| | | |
|---|---|---|
| casp-3 small for | TTCCATATGAGTGGTGTTGATGATGACATGGCG | SEQ ID NO:92 |
| casp-3 small rev | AAGGAATTCTTAGTGATAAAAATAGAGTTCTTTTGTGAG | SEQ ID NO:93 |

Amplified DNA corresponding to either the large subunit or the small subunit of caspase-3 was then cleaved with the restriction enzymes EcoRI and NdeI and directly cloned using standard molecular biology techniques into pRSET-b (Invitrogen) digested with EcoRI and NdeI. [See e.g., Tewari. M., et al., Yama/CPP32 beta, a mammalian homolog of CED-3, is a CrmA-inhibitable protease that cleaves the death substrate poly (ADP-ribose) polymerase, Cell 81: 801-809 (1995)].

Generation of Casp-3 Cys Mutations

Plasmids containing DNA encoding either the large or small subunits of Caspase-3 were separately transformed into E. coli K12 CJ236 cells (New England BioLabs) and cells containing each construct were selected by their ability to grow on ampicillin containing agar plates. Overnight cultures of the large and small subunits were individually grown in 2YT (containing 100 μg/mL of ampicillin) at 37° C. Each culture was diluted 1:100 and grown to A₆₀₀=0.3-0.6. A 1.5 mL sample of each culture was removed and infected with 10 μL of phage VCS-M13 (Stratagene), shaken at 37° C. for 60 minutes, and an overnight culture of each was prepared with 1 mL of the infected culture diluted 1:100 in 2YT with 100 μg/mL of ampicillin and 20 μg/ml of chloramphenicol and grown at 37° C. Cells were centrifuged at 3000 rcf for 10 minutes and ⅕ volume of 20%PEG/2.5 M NaCl was added to the supernatant. Samples were incubated at room temperature for 10 minutes and then centrifuged at 4000 rcf for 15 minutes. The phage pellet was resuspended in PBS and spun at 15 K rpm for 10 minutes to remove remaining particulate matter. Supernatant was retained, and single stranded DNA was purified from the supernatant following procedures for the QIA prep spin M13 kit (Qiagen).

Mutagenic Oligonucleotides

Cysteine mutations in the small subunit were made with the corresponding primers:

| | | |
|---|---|---|
| Y204C | TCGCCAGAACAATAACCAGG | SEQ ID NO:94 |
| S209C | GCCATCCTTACAATTTCGCCA | SEQ ID NO:95 |
| W214C | CTGGATGAAACAGGAGCCATC | SEQ ID NO:96 |
| S251C | AGCCTCAAAGCAAAAGGACTC | SEQ ID NO:97 |
| F256C | CTTTGCATGACAAGTAGCGTC | SEQ ID NO:98 |

Cysteine mutations in the large subunit were made with the corresponding primers:

| | | |
|---|---|---|
| M61C | CCGAGATGTACATCCAGTGCT | SEQ ID NO:99 |
| T62C | AGACCGAGAACACATTCCAGT | SEQ ID NO:100 |
| S65C | ATCTGTACCACACCGAGATGT | SEQ ID NO:101 |
| H121C | TTCTTCACCACAGCTCAGAAG | SEQ ID NO:102 |
| L168C | GCCACAGTCACATTCTGTACC | SEQ ID NO:103 |

Approximately 100 pmol of each primer was phosphorylated by incubating at 37° C. for 60 minutes in buffer containing 1×TM Buffer (0.5 M Tris pH 7.5, 0.1 M MgCl₂), 1 mM ATP, 5 mM DTT, and 5U T4 Kinase (NEB). Kinased primers were annealed to the template DNA in a 20 μL reaction volume (˜50 ng kinased primer, 1×TM Buffer, and 10-50 ng single-stranded DNA) by incubation at 85° C. for 2 minutes, 50° C. for 5 minutes, and then at 4° C. for 30-60 minutes. An extension cocktail (2 mM ATP, 5 mM dNTPs, 30 mM DTT, T4 DNA ligase (NEB), and T7 polymerase (NEB)) was added to each annealing reaction and incubated at room temperature for 3 hours. Mutagenized DNA was transformed into E. coli XL1-Blue cells, and colonies containing plasmid DNA selected were for by growth on LB agar plates containing 100 μg/ml ampicillin. DNA sequencing was used to identify plasmids containing the appropriate mutation.

Expression of Casp-3 Mutant Proteins

Plasmid DNA encoding cysteine mutations in the large subunit were transformed into Codon Plus BL21 Cells and plasmid DNA encoding cysteine mutations in the small subunit were transformed into BL21 (DE3) pLysS Cells. Codon Plus BL21 Cells containing plasmids encoding wild-type and cysteine mutated versions of the large subunit were grown in 2YT containing 150 μg/mL of ampicillin overnight at 37° C. and immediately harvested. BL21 pLysS cells containing plasmids encoding wild-type and cysteine mutated versions of the small subunit were grown in 2YT at 37° C. with 150 μg/mL of ampicillin until A₆₀₀=0.6. Cultures were subsequently induced with 1 mM IPTG and grown for an additional 3-4 hours at 37° C. After harvesting cells by centrifuging at 4K rpm for 10 minutes, the cell pellet was resuspended in Tris-HCl (pH 8.0)/5 mM EDTA and micro fluidized twice. Inclusion bodies were isolated by centrifugation at 9K rpm for 10 minutes and then resuspended in 6 M guanidine hydrochloride. Denatured subunits were rapidly and evenly diluted to 100 μg/mL in renaturation buffer (100 mM Tris/KOH (pH 8.0), 10% sucrose, 0.1% CHAPS, 0.15 M NaCl, and 10 mM DTT) and allowed to renature by incubation at room temperature for 60 minutes with slow stirring.

Renatured proteins were dialyzed overnight in buffer containing 10 mM Tris (pH 8.5), 10 mM DTT, and 0.1 mM EDTA. Precipitate was removed by centrifuging at 9K rpm for 15 minutes and filtering the supernatant through a 0.22 μm cellulose nitrate filter. The supernatant was then loaded onto an anion-exchange column (Uno5 Q-Column (BioRad)), and correctly folded caspase-3 protein was eluted with a 0-0.25 M NaCl gradient at 3 mL/min. Aliquots of each fraction were electrophoresed on a denaturing polyacrylamide gel and fractions containing Caspase-3 protein were pooled.

### EXAMPLE 7

### Cloning and Mutagenesis of Human Protein Tyrosine Phosphatase-1B (PTP-1B)

PTP-1B (accession number SWS P18031) is a tyrosine phosphatase that has a C-terminal domain that is associated to the endoplasmic reticulum (ER) and a phosphatase domain that faces the cytoplasm. The proteins that it dephosphorylates are transported to this location by vesicles. The activity of PTP-1B is regulated by phosphorylation on serine and protein degradation. PTP-1Bis a negative regulator of insulin signaling, and plays a role in the cellular response to interferon stimulation. This phosphatase may play a role in obesity by decreasing the sensitivity of organisms to leptin, thereby increasing appetite. Additionally, PTP-1B plays a role in the control of cell growth. A crystal structure has been solved for PTP-1B [1PTY, Puius, Y. A., et al., Proc Natl Acad Sci USA 94: 13420-13425 (1997)].

Cloning of human PTP-1B

Full length human PTP-1B is 435 amino acids in length; the protease domain comprises the first 288 amino acids. Because truncated portions of PTP-1B comprising the protease domain is fully active, various truncated versions of PTP-1B are often used. A cDNA encoding the first 321 amino acids of human PTP-1B was isolated from human fetal heart total RNA (Clontech). Oligonucleotide primers corresponding to nucleotides 91 to 114 (For) and complementary to nucleotides 1030 to 1053 (Rev) of the PTP-1B cDNA [Genbank M31724.1, Chernoff, J., et al., Proc. Natl. Acad. Sci. U.S.A. 87: 2735-2739 (1990)] were synthesized and used to generate a DNA using the polymerase chain reaction.

| SEQ ID NO:104 | |
|---|---|
| Forward | GCCCATATGGAGATGGAAAAGGAGTTCGAG |
| SEQ ID NO:105 | |
| Rev | GCGACGCGAATTCTTAATTGTGTGGCTCCAGGATTCGTTT |

The primer Forward incorporates an NdeI restriction site at the first ATG codon and the primer Rev inserts a UAA stop codon followed by an EcoRI restriction site after nucleotide 1053. cDNAs were digested with restriction nucleases NdeI and EcoRI and cloned into pRSETc (Invitrogen) using standard molecular biology techniques. The identity of the isolated cDNA was verified by DNA sequence analysis (methodology is outlined in a later paragraph).

A shorter cDNA, PTP-1B 298, encoding amino acid residues 1-298 was generated using oligonuclotide primers Forward and Rev2 and the clone described above as a template in a polymerase chain reaction.

Rev2 TGCCGGAATTCCTTAGTCCTCGTGGGAAAGCTCC SEQ ID NO: 106

Generation of PTP-1B Cysteine Mutants

Site-directed mutants of PTP-1B (amino acids 1-321), PTP-1B 298 (amino acids 1-298) and PTP-1B 298-2M (with Cys32 and Cys92 changed to Ser and Val, respectively) were prepared by the single-stranded DNA method (modification of Kunkel, 1985). 298-2M was made with the following oligonucleotides.

| | | |
|---|---|---|
| C32S | CTTGGCCACTCTAGATGGGAAGTCACT | SEQ ID NO:107 |
| C92V | CCAAAAGTGACCGACTGTGTTAGGCAA | SEQ ID NO:108 |

Oligonucleotides were designed to contain the desired mutations and 12 bases of flanking sequence on each side of the mutation. The single-stranded form of the PTP-1B/pRSET, PTP-1B 298/pRSET and PTP-1B 298-2M/pRSET plasmid was prepared by transformation of double-stranded plasmid into the CJ236 cell line (1 μl double-stranded plasmid DNA, 2 μl 5×KCM salts, 7 μl water, 10 μl PEG-DMSO competent CJ236 cells; incubated on ice for 20 minutes followed by 25° C. for 10 minutes; plated on LB/agar with 100 μg/ml ampicillin and incubated at 37° C. overnight). Single colonies of CJ236 cells were then grown in 100 ml 2YT media to midlog phase; 5 μl VCS helper phage (Stratagene) were then added and the mixture incubated at 37° C. overnight. Single-stranded DNA was isolated from the supernatant by precipitation of phage (⅕ volume 20% PEG 8000/2.5M NaCl; centrifuge at 12K for 15 minutes). Single-stranded DNA was then isolated from phage using Qiagen single-stranded DNA kit.

Site-directed mutagenesis was accomplished as follows. Oligonucleotides were dissolved in TE (10 mM Tris pH 8.0, 1 mM EDTA) to a concentration of 10 OD and phosphorylated on the 5′ end (2 μl oligonucleotide, 2 μl 10 mM ATP, 2 μl 10×Tris-magnesium chloride buffer, 1 μl 100 mM DTT, 12.5 μl water, 0.5 μl T4 PNK; incubate at 37° C. for 30 minutes). Phosphorylated oligonucleotides were then annealed to single-stranded DNA template (2 μl single-stranded plasmid, 0.6 μl oligonucleotide, 6.4 μl water; heat at 94° C. for 2 minutes, slow cool to room temperature). Double-stranded DNA was then prepared from the annealed oligonucleotide/template (add 2 μl 10×TM buffer, 2 μl 2.5 mM dNTPs, 1 μl 100 mM DTT, 0.5 μl 10 mM ATP, 4.6 μl water, 0.4 μl T7 DNA polymerase, 0.2 μl T4 DNA ligase; incubate at room temperature for two hours). E. coli (XL1 blue, Stratagene) were then transformed with the double-stranded DNA (5 μl double-stranded DNA, 5 μl 5×KCM, 15 μl water, 25 μl PEG-DMSO competent cells; incubate 20 minutes on ice, 10 min. at room temperature), plated onto LB/agar containing 100 μg/ml ampicillin, and incubated at 37° C. overnight. Approximately four colonies from each plate were used to inoculate 5 ml 2YT containing 100 μg/ml ampicillin; these cultures were grown at 37° C. for 18-24 hours. Plasmids were then isolated from the cultures using Qiagen miniprep kit. These plasmids were sequenced to determine which clones contained the desired mutation.

A listing of the constructs and the single mutations to cysteine made in each context is given below.

| Construct | Mutants |
|---|---|
| PTP-1B 321 | H25C, D29C, R47C, D48C, S50C, K120C, M258C |
| PTP-1B 298 | H25C, D29C, D48C, S50C, K120C, M258C, F280C |
| PTP-1B 298-2M | E4C, E8C, H25C, A27C, D29C, K36C, Y46C, R47C, |
| | D48C, V49C, S50C, F52C, K120C, S151C, Y152C, |
| | T178C, D181C, F182C, E186C, S187C, A189C, |
| | K197C, E200C, L272C, E276C, I218C, M258C, |
| | Q262C, V287C |

However, it should be understood that any of the site-directed mutants may be made in any construct of PTP-1B. For example, another construct is another truncated version of PTP-1B having residues 1-382, shown as SEQ ID NO: 109 below.

| | |
|---|---|
| 1 | MEMEKEFEQI DKSGSWAAIY QDIRHEASDF PCRVAKLPKN KNRNRYRDVS PFDHSRIKLH |
| 61 | QEDNDYINAS LIKMEEAQRS YILTQGPLPN TCGHFWEMVW EQKSRGVVML NRVMEKGSLK |
| 121 | CAQYWPQKEE KEMIFEDTNL KLTLISEDIK SYYTVRQLEL ENLTTQETRE ILHFHYTTWP |
| 181 | DFGVPESPAS FLNFLFKVRE SGSLSPEHGP VVVHCSAGIG RSGTFCLADT CLLLMDKRKD |
| 241 | PSSVDIKKVL LEMRKFRMGL IQTADQLRFS YLAVIEGAKF IMGDSSVQDQ WKELSHEDLE |
| 301 | PPPEHIPPPP RPPKRILEPH NGKCREFFPN HQWVKEETQE DKDCPIKEEK GSPLNAAPYG |
| 361 | IESMSQDTEV RSRVVGGSLR GA |

Mutagenic Oligonucleotides

| | | |
|---|---|---|
| E4C | CTCGAACTCCTTGCACATCTCCATATG | SEQ ID NO:110 |
| E8C | CTTGTCGATCTGGCAGAACTCCTTTTC | SEQ ID NO:111 |
| H25C | GTCACTGGCTTCACATCGGATATCCTG | SEQ ID NO:112 |
| A27C | TGGGAAGTCACTGCATTCATGTCGGAT | SEQ ID NO:113 |
| D29C | TCTACATGGGAAGCAACTGGCTTCATG | SEQ ID NO:114 |
| K36C | GTTCTTAGGAAGACACGCCACTCTACA | SEQ ID NO:115 |
| Y46C | ACTGACGTCTCTGCACCTATTTCGGTT | SEQ ID NO:116 |
| R47C | GGGACTGACGTCACAGTACCTATTTCG | SEQ ID NO:117 |
| D48C | AAAGGGACTGACGCATCTGTACCTATT | SEQ ID NO:118 |
| V49C | GTCAAAGGGACTGCAGTCTCTGTACCT | SEQ ID NO:119 |
| S50C | CTATGGTCAAAGGGACAGACGTCTCTGTACC | SEQ ID NO:120 |
| F52C | CCGACTATGGTCACAGGGACTGACGTC | SEQ ID NO:121 |
| K120C | GTATTGTGCGCAACATAACGAACCTTT | SEQ ID NO:122 |
| 5151C | CACTGTATAATAGCACTTGATATCTTC | SEQ ID NO:123 |
| Y152C | GTCGCACTGTATAACATGACTTGATATC | SEQ ID NO:124 |
| T178C | CAAAGTCAGGCCAGCAGGTATAGTGGAA | SEQ ID NO:125 |
| D181C | AGGGACTCCAAAGCAAGGCCATGTGGT | SEQ ID NO:126 |
| E186C | GAATGAGGCTGGTGAGCAAGGGACTCCAAAG | SEQ ID NO:127 |
| S187C | GAATGAGGCTGGGCATTCAGGGACTCC | SEQ ID NO:128 |
| A189C | GTTCAAGAATGAGCATGGTGATTCAGG | SEQ ID NO:129 |
| K197C | CTGACTCTCGGACGCAGAAAAGAAAGTTC | SEQ ID NO:130 |
| E200C | GAGTGACCCTGAGCATCGGACTTTGAAAAG | SEQ ID NO:131 |
| M258C | CTGGATCAGCCCACACCGAAACTTCCT | SEQ ID NO:132 |
| Q262C | CTGGTCGGCTGTACAGATCAGCCCCAT | SEQ ID NO:133 |
| L272C | CTTCGATCACAGCGCAGTAGGAGAACCG | SEQ ID NO:134 |
| E276C | GAATTTGGCACCGCAGATCACAGCCAG | SEQ ID NO:135 |
| 1281C | AGAGTCCCCCATGCAGAATTTGGCACC | SEQ ID NO:136 |
| V287C | CCACTGATCCTGGCAGGAAGAGTCCCC | SEQ ID NO:137 |

Besides mutations to cysteines, mutations removing naturally occurring cysteines can also be made. For example, two different “scrubs” of Cys215 were made in the PTP-1B 298-2M context using the following oligonucleotides:

| | | |
|---|---|---|
| C215A | GATGCCTGCACTGCCGTGCACCACAAC | SEQ ID NO:138 |
| C215S | GATGCCTGCACTGGAGTGCACCACAAC | SEQ ID NO:139 |

In the PTP-1B 298 context, two quadruple mutants were made using the C92A oligonucleotide shown below. They are C32S, C92A, V287C, C215A, which used SEQ ID NO: 107 SEQ ID NO: 140 SEQ ID NO: 137 and SEQ ID NO: 138 and C32S, C92A, E276C, C215A, which used SEQ ID NO: 107, SEQ ID NO: 140 SEQ ID NO: 135 and SEQ ID NO: 138.

C92A CCAAAAGTGACCGGCTGTGTTAGGCAA SEQ ID NO: 140

Sequencing of PTP-1B clones was accomplished as follows. The concentration of plasmid DNA was quantitated by absorbance at 280 nm. 1000 ng of plasmid was mixed with sequencing reagents (1 μg DNA, 6 μl water, 1 μl sequencing primer at 3.2 pm/μl, 8 μl sequencing mixture with Big Dye [Applied Biosystems]). The sequencing primers are SEQ ID NO: 17 and SEQ ID NO: 18. The mixture was then run through a PCR cycle (96° C., 10 s; 50° C., 5 s; 60° C. 4 minutes; 25 cycles) and the DNA reaction products were precipitated (20 μl mixture, 80 μl 75% isopropanol; incubated 20 minutes at room temperature then pelleted at 14 K rpm for 20 minutes; wash with 250 μl 75% isopropanol; heat 1 minute at 94° C.). The precipitated products were then resuspended in 20 μl TSB (Applied Biosystems) and the sequence read and analyzed by an Applied Biosystems 310 capillary gel sequencer. In general, ¼ of the plasmids contained the desired mutation.

Expression of Cysteine Mutants of PTP-1B

Mutant proteins were expressed as follows. PTP-1B clones were transformed into BL21 codon plus cells (Stratagene) (1 μl double-stranded DNA, 2 μl 5×KCM, 7 μl water, 10 μl DMSO competent cells; incubate 20 minutes at 4° C., 10 minutes at room temperature), plated onto LB/agar containing 100 μg/ml ampicillin, and incubated at 37° C. overnight. 2 single colonies were picked off the plates or from frozen glycerol stocks of these mutants and inoculated in 100 ml 2YT with 50 μg/ml carbenicillin and grown overnight at 37° C. 50 ml from the overnight cultures were added to 1.5 L of 2YT/carbenicillin (50 μg/ml) and incubated at 37° C. for 3-4 hours until late-log phase (absorbance at 600 nm˜0.8-0.9). At this point, protein expression was induced with the addition of IPTG to a final concentration of 1 mM. Cultures were incubated at 37° C. for another 4 hours and then cells were harvested by centrifugation (7K rpm, 7 minutes) and frozen at −20° C.

PTP-1B proteins were purified from the frozen cell pellets as described in the following. First, cells were lysed in a microfluidizer in 100 ml of buffer containing 20 mM MES pH 6.5, 1 mM EDTA, 1 mM DTT, and 10% glycerol buffer (with 3 passes through a Microfluidizer [Microfluidics 110S]) and inclusion bodies were removed by centrifugation (10K rpm, 10 minutes). Purification of all PTP-1B mutants was performed at 4° C. The supernatants from the centrifugation were filtered through 0.45 μm cellulose acetate (5 μl of this material was analyzed by SDS-PAGE) and loaded onto an SP Sepharose fast flow column (2.5 cm diameter×14 cm long) equilibrated in Buffer A (20 mM MES pH 6.5, 1 mM EDTA, 1 mM DTT, 1% glycerol) at 4 ml/min.

The protein was then eluted using a gradient of 0-50% Buffer B over 60 minutes (Buffer B: 20 mM MES pH 6.5, 1 mM EDTA, 1 mM DTT, 1% glycerol, 1 M NaCl). Yield and purity was examined by SDS-PAGE and, if necessary, PTP-1B was further purified by hydrophobic interaction chromatography (HIC). Protein was supplemented with ammonium sulfate until a final concentration of 1.4 M was reached. The protein solution was filtered and loaded onto an HIC column at 4 ml/min in Buffer A2: 25 mM Tris pH 7.5, 1 mM EDTA, 1.4 M (NH₄)₂SO₄, 1 mM DTT. Protein was eluted with a gradient of 0-100% Buffer B over 30 minutes (Buffer B2: 25 mM Tris pH 7.5, 1 mM EDTA, 1 mM DTT, 1% glycerol). Finally, the purified protein was dialyzed at 4° C. into the appropriate assay buffer (25 mM Tris pH 8, 100 mM NaCl, 5 mM EDTA, 1 mM DTT, 1% glycerol). Yields varied from mutant to mutant but typically were within the range of 3-20 mg/L culture.

### EXAMPLE 8

### Cloning and Mutagenesis of Human Immunodeficiency Virus Integrase (HIV IN)

HIV IN is one of three key enzyme targets of the human immunodeficiency virus; it removes two nucleotides from each 3′ end of the originally blunt viral DNA, and inserts the viral DNA into the host DNA by strand transfer. The integration process is completed by host DNA repair enzymes. HIV IN has three distinct domains: the N-terminal domain, the catalytic core domain, and the C-terminal domain. Although the X-ray crystal structures of each of these isolated domains have been solved, it is not yet clear how they interact with each other. Integration is absolutely essential for the replication of the virus and progression of disease, and thus integrase inhibitors can be used in the treatment of HIV/AIDS. Structures of core domain of integrase are available [1EXQ, Chen, J. C. -H., et al., Proc. Natl. Acad. Sci. U.S.A. 97: 8233-8238 (2000); 1BL3, Maignan, S., et al., J Mol Biol 282:359-368 (1998); in complex with tetraphenyl arsonium, 1HYZ and 1HYV, Molteni, V., et al., Acta Crystallogr D Bio Crystallog., 57:536-544 (2001)].

Cloning of HIV IN

Numbering of the wild type and mutant HIV-1 integrase residues follows the convention of the first amino acid residue of the mature protein being residue number 1, and the HIV-1 integrase catalytic core domain being comprised of residues 52-210 [Leavitt, A. D., et al., J Biol Chem 268: 2113-2119 (1993)].

A plasmid construct, pT7-7 HT-INₜₑₜᵣₐ, encoding the HIV integrase core domain (residues 50-212), having an N-terminal 6×histidine tag and thrombin cleavable linker, and C56S, W131D, F139D, and F185K mutations in the pT7-7 (Novagen) vector background [Chen, J. C. -H., et al., Proc. Natl. Acad. Sci. U.S.A. 97: 8233-8238 (2000)] was obtained from Dr. Andy Leavitt at UCSF. Upon comparison of the crystal structure of this core domain variant [Chen, J. C. -H., et al., Proc. Natl. Acad. Sci. U.S.A. 97: 8233-8238 (2000)] to other integrase core structures, it was noted that the F139D mutation, designed to increase solubility of the protein, caused a rotation of the side chain that transmitted a distortion to the catalytically important Asp116. The mutation was therefore reverted to the wild-type phenylalanine residue by Quickchange mutagenesis (Stratagene), following manufacturer's instructions and using SEQ ID NO: 141 and SEQ ID NO: 142.

| | |
|---|---|
| D139F1-int | |
| GTATCAAACAGGAATTCGGTATCCCGTACAAC | SEQ ID NO:141 |
| D139F2-int | |
| GTTGTACGGGATACCGAATTCCTGTTTGATACC | SEQ ID NO:142 |

This generated pT7-7 HT-INₜᵣᵢ, encoding the triple mutant (C56S, W131D, F185K) of the integrase core, SEQ ID NO: 143.

| | |
|---|---|
| 52 | GQVDSSPGIW QLDCTHLEGK VILVAVHVAS GYIEAEVIPA ETGQETAYFL LKLAGRWPVK |
| 112 | TIHTDNGSNF TGATVRAACD WAGIKQEFGI PYNPQSQGVV ESMNKELKKI IGQVRDQAEH |
| 172 | LKTAVQMAVF IHNKKRKGGI GGYSAGERIV DIIATDIQT |

In preparation for making cysteine mutations at tethering sites, the two wild-type cysteines, (C130 and C65) were replaced by alanine residues and the DNA encoding the His-tagged INₜᵣᵢ core domain transferred into the pRSET A vector, containing an F1 origin of replication that allows preparation of single-stranded plasmid DNA, and thus mutagenesis by the Kunkel method [Kunkel, T. A., et al., Methods Enzymol. 204: 125-139 (1991)]. Replacement of C130 by alanine was accomplished by cassette mutagenesis, using the double stranded cassette composed of SEQ ID NO: 144 and SEQ ID NO: 145. The cassette, containing the appropriate overhangs at each end, was ligated into pT7-7 HT-INₜᵣᵢ digested with BsiWI and EcoRI.

| | | |
|---|---|---|
| C130A cassette 1 | GTACGTGCTGCAGCCGACTGGGCTGGTATCAAACAGG | SEQ ID NO:144 |
| C130A cassette 2 | GAATTCCTGTTTGATACCAGCCCAGTCGGCTGCAGCAC | SEQ ID NO:145 |

The C65A mutation was carried out independently by Quickchange mutagenesis on pT7-7 HT-INₜᵣᵢ using SEQ ID NO: 146 and SEQ ID NO: 147.

| | |
|---|---|
| C65A1-int | |
| ATCTGGCAACTGGACGCGACTCACCTCGAGGGT | SEQ ID NO:146 |
| C65A2-int | |
| ACCCTCGAGGTGAGTCGCGTCCAGTTGCCAGAT | SEQ ID NO:147 |

The DNA encoding HT-C130A integrase core domain was subcloned into the pRSET A vector by PCR cloning. SEQ ID NO: 148 and SEQ ID NO: 149 were used as PCR primers, and the resulting amplified product was digested with NdeI and Hind III, and ligated into pRSET A that had been digested with the same enzymes, to generate pRSET-HT-C130A-INₜᵣᵢ.

| | |
|---|---|
| C130_rsetF | |
| GGAGATATACATATGCACCACCATCACC | SEQ ID NO:148 |
| C130_rsetR | |
| ATCATCGATGATAAGCTTCCTAGGTCTGG | SEQ ID NO:149 |

A BamHI fragment of pT7-7 HT-C65A-INₜᵣᵢ containing the C65A mutation was ligated into pRSET-HT-C130A-INₜᵣᵢ, to generate pRSET-HT-INₜₑₘₚₗₐₜₑ. This plasmid served as a template for further Kunkel mutagenesis to introduce cysteine substitutions at positions chosen for tethering. SEQ ID NO: 17 was used for sequencing.

Mutagenic Oligonucleotides

| | | |
|---|---|---|
| Q62C | GTGAGTCGCGTCCAGGCACCAGATACCCGG | SEQ ID NO:150 |
| D64C | CTCGAGGTGAGTCGCGCACAGTTGCCAGATAC | SEQ ID NO:151 |
| T66G | CTTTACCCTCGAGGTGACACGCGTCCAGTTGCC | SEQ ID NO:152 |
| H67C | GGATAACTTTACCCTCGAGGCAAGTCGCGTCCAGTTG | SEQ ID NO:153 |
| L68C | AACTTTACCCTCGCAGTGAGTCGCGTCCA | SEQ ID NO:154 |
| K71C | GCAACCAGGATAACGCAACCCTCGAGGTG | SEQ ID NO:155 |
| E92C | CAGTTTCCTGACCAGTGCAGGCCGGGATAACTTC | SEQ ID NO:156 |
| H114C | GGATCCGTTOTCAGTGCAGATGGTTTTAACCGGC | SEQ ID NO:157 |
| D116C | GTTGGATCCGTTGCAAGTGTGGATGGTTTTAACCG | SEQ ID NO:158 |
| N120C | CGGTAGCACCAGTGAAGCAGGATCCGTTGTCAGTG | SEQ ID NO:159 |
| N144C | CACCCTGAGACTGCGGGCAGTACGGGATACCGA | SEQ ID NO:160 |
| Q148C | CATAGATTCAACAACACCGCAAGACTGCGGGTTGT | SEQ ID NO:161 |
| I151C | GCTCTTTGTTCATAGATTCGCAAACACCCTGAGA | SEQ ID NO:162 |
| E152C | GCTCTTTGTTCATAGAGCAAACAACACCCTGAGA | SEQ ID NO:163 |
| N155C | CCGATGATTTTTTTGAGCTCTTTGCACATAGATTCAACAAC | SEQ ID NO:164 |
| K156C | CCGATGATTTTTTTGAGCTCGCAGTTCATAGATTC | SEQ ID NO:165 |
| K159C | CCTGACCGATGATTTTGCAGAGCTCTTTGTTCAT | SEQ ID NO:166 |
| G163C | CCTGATCACGAACCTGGCAGATGATTTTTTTG | SEQ ID NO:167 |
| Q168C | GGTTTTCAGGTGTTCAGCGCAATCACGAACCTGA | SEQ ID NO:168 |
| T174C | GCCATCTGAACCGCGCATTTCAGGTGTTCAGCC | SEQ ID NO:169 |

Expression of IN Cysteine Mutants

pT7-7 and pRSET integrase core domain expression plasmids were transformed into BL21 star E. coli (Invitrogen) by standard methods, and a single colony from the resulting plate was used to inoculate 250 mL of 2×YT broth containing 100 μg/mL ampicillin. Following overnight growth at 37° C., the cells were harvested by centrifugation at 4K rpm and resuspended in 100 mL 2YT/amp. 40 mL of the washed cells was used to inoculate 1.5 L of the same media, and after growth at 37° C. to an OD at 600 nm of between 0.5 and 0.8, the culture was moved to 22° C. and allowed to cool. IPTG was added to a final concentration of 0.1 mM and expression continued 17-19 h at 22° C. Cells were harvested by centrifugation at 4K rpm. Cell pellets were resuspended in 100 mL Wash 5 buffer (Wash 5: 20 mM Tris-HCl, 1 M MgCl₂, 5 mM imidazole, 5 mM β-mercaptoethanol, pH 7.4) and lysis was accomplished by sonication for 1 minute, repeated a total of 3 times with 2 minutes rest between. Cell debris was removed by centrifugation at 14K rpm followed by filtration. Integrase core domain was purified by affinity chromatography on Ni-NTA superflow resin (Qiagen) at 4° C. After loading the cell lysate, the column was washed with Wash 40 buffer (Wash 40: 20 mM Tris-HCl, 0.5 M NaCl, 40 mM imidazole, 5 mM β-mercaptoethanol, pH 7.4) and His-tagged IN core domain eluted with E400 buffer (E400: 20 mM Tris-HCl, 0.5 M NaCl. 400 mM imidazole, 5 mM β-mercaptoethanol). The purified enzyme was dialyzed versus 20 mM Tris, 0.5 M NaCl, 2.5 mM CaCl₂, 5 mM β-mercaptoethanol, pH 7.4 at 4° C., and aliquoted into 1.5 mL tubes. Biotinylated thrombin (Novagen) (2U thrombin/mg of protein) was added and the tubes rotated overnight at 4° C., followed by thrombin removal using streptavidin-agarose resin (Novagen) and separation of His-tagged protein and peptides from the cleaved material by passage through a second column of Ni-NTA sepharose fast-flow. Purified, cleaved integrase core domain was dialyzed against 20 mM Tris-HCl, 0.5 M NaCl, 3 mM DTT, and 5% glycerol, pH 7.4, and stored at −20° C. Protein concentrations were determined by absorbance at 280 nm after desalting on NAP-5 columns (Pharmacia), using ε₂₈₀^{1%}=(1.174), and molecular weights confirmed by ESI mass spectrometry (Finnigan).

### EXAMPLE 9

### Human Beta-Site Amyloid Precursor Protein Cleaving Enzyme1 (BACE1)

BACE1 (accession number SWS 56817) is a type1 integral glycoprotein that is an aspartic protease. Found mostly in the Golgi, BACE1 cleaves the amyloid precursor protein to form the Abeta peptide. A strong association has been shown between deposition of this peptide on the cerebrum and Alzheimer's disease; therefore BACE1 is one of the primary targets for this disease. A crystal structure of BACE1 has been solved [1FKN, Hong, L. et al., Science 290:150-153 (2000)].

Cloning of Human BACE1

The proprotease domain gene sequence (bases 64-1362, amino acid residues 22-454) was subcloned from pFBHT into the E. coli expression vector pRSETC by PCR, to create pB22, which served as a template for mutagenesis to incorporate cysteine tethering sites. For a description of pFBHT, a modified pFastBac plasmid, see example 4 above. The subcloning was accomplished as follows. The cDNA encoding full-length human BACE1, bases 1-1551, starting from the initiator Met codon and including an extra 48 bases of mRNA transcript following the stop codon [Vassar, R., et al., Science 286: 735-741 (1999)] was obtained by a combination of PCR cloning of the 3′ 1425 bases from human cDNA libraries, and synthesis of the remaining 5′ 126 bases by serial overlapping PCR. All PCR reactions were performed using Advantage2 polymerase (Clontech) according to manufacturers instructions. A fragment spanning bases 126-374 was obtained by PCR from a human cerebral cortex library and SEQ ID NO: 170 and SEQ ID NO: 171; a fragment spanning bases 339-770 was obtained by PCR from a Stratagene Unizap XR human brain cDNA library, and SEQ ID NO: 172 and SEQ ID NO: 173; and the 3′ end fragment, spanning bases 735-1551, was obtained by PCR from a human brain library, using SEQ ID NO: 174 and SEQ ID NO: 175. The three fragments, having 35 bp of overlap at the junctions, were gel purified and combined in one PCR reaction, using primers to the ends (SEQ ID NO: 170 and SEQ ID NO: 176) to amplify the 126-1551 product.

| | | |
|---|---|---|
| For2 | GCTGCCCCGGGAGACCGACGAAGA | SEQ ID NO:170 |
| midRev2 | CGGAGGTCCCGGTATGTGCTGGAC | SEQ ID NO:171 |
| midFor | CCAGAGGCAGCTGTCCAGCACATA | SEQ ID NO:172 |
| midRev1 | TCCCGCCGGATGGGTGTATACCAG | SEQ ID NO:173 |
| BACE14 | GTACACAGGCAGTCTCTGGTATACACC | SEQ ID NO:174 |
| BACE11 | GTGTGGTCCAGGGGAATCTCTATCTTCTG | SEQ ID NO:175 |
| BACE5 | GTCATCGTCTCGAGTCACTTCAGCAGGGAGATGTCATCAG | SEQ ID NO:176 |

The 126-1551 piece, and the subsequent elongated products, were used as a templates for serial overlapping PCR reactions, to add the remaining 5′-126 bases using SEQ ID NO: 177, SEQ ID NO: 178 and SEQ ID NO: 179 as forward primers, with SEQ ID NO: 176 always at the reverse primer.

| | |
|---|---|
| BACE fill2 | |
| CGGCTGCCCCTGCGCAGCGGCCTGGGGGGCGCCCCCCTGGGGCTGCGGCTGCCCCGGGAG | SEQ ID NO:177 |
| BACE fill1 | |
| ATGGGCGCGGGAGTGCTGCCTGCCCACGGCACCCAGCACGGCATCCGGCTGCCCCTGCGC | SEQ ID NO:178 |
| BACE for-EcoRI | |
| CCGGAATTCATGGCCCAAGCCCTGCCCTGGCTCCTGCTGTGGATGGGCGCGGGAGTG | SEQ ID NO:179 |

SEQ ID NO: 179 and SEQ ID NO: 176 contained EcoRI and XhoI restriction sites, respectively, and digestion of the PCR product, along with the Baculovirus expression vector, pFBHT, with the same enzymes was followed by gel purification and ligation of the resulting DNA fragments, yielding the construct, pFBHT-BACE. This construct was used as a template for PCR amplification of bases 1-1362, corresponding to the preproBACE soluble protease domain, using SEQ ID NO: 180 and SEQ ID NO: 181.

| | |
|---|---|
| proFor-Nde | |
| CGCCATATGGCGGGAGTGCTGCCTGCCCACGGC | SEQ ID NO:180 |
| BACErev-RI | |
| CCGGAATTCTCAGGTTGACTCATCTGTCTGTGGAAT | SEQ ID NO:181 |

SEQ ID NO: 180 and SEQ ID NO: 181 contained NdeI and EcoRI restriction sites, respectively, and digestion of the PCR product, along with the E. coli expression vector, pRSETC, with the same enzymes was followed by gel purification and ligation of the resulting DNA fragments led to the construct pB1. Vector pB1 was then used as a template for Kunkel mutagenesis (Kunkel, T. A., et al., Methods Enzymol. 154:367-382 [1987]) to delete the BACE presequence (bases 1-63), producing the construct pB22. pB22 served as a template for mutagenesis to incorporate cysteine tethering sites, using either the Kunkel method or a Quickchange mutagenesis kit (Stratagene).

Mutagenenic Oligonucleotides

| | | |
|---|---|---|
| L91C | GCCTGTATCCACGCAGATGTTGAGCGT | SEQ ID NO:182 |
| T133C | CTTGCCCTGGCAGTAGGGCACATACCA | SEQ ID NO:183 |
| Q134C | TTCCCACTTGCCGCAGGTGTAGGGCAC | SEQ ID NO:184 |
| F169C | CGTTGATGAAGCACTTGTCTGATTCGC | SEQ ID NO:185 |
| I171C | GTTGGAGCCGTTGCAGAAGAACTTGTC | SEQ ID NO:186 |
| R189C | GGAGTCGTCAGGACAGGCAATCTCAGC | SEQ ID NO:187 |
| Y259C | GATGACCTCATAACACCACTCCCGCCG | SEQ ID NO:188 |
| N294C | GGGCAAACGAAGGCAGGTGGTGCCACT | SEQ ID NO:189 |
| R296C | TTTCTTGGGCAAACAAAGGTTGGTGGT | SEQ ID NO:190 |
| T390C | CATAACAGTGCCGCAGGATGACTGTGA | SEQ ID NO:191 |
| V393C | AACAGCTCCCATACAAGTGCCCGTGGA | SEQ ID NO:192 |

Expression of Human BACE1 Mutants

pB22 was transformed into BL21star E. coli (Invitrogen) by standard methods, and a single colony from the resulting plate was used to inoculate 50 mL of 2×YT broth containing 100 μg/mL ampicillin. Following overnight growth at 37° C., 40 mL of the culture was used to inoculate 1.5 L of the same media, and after growth at 37° C. to an OD at 600 nm of between 0.5 and 0.8, IPTG was added to a final concentration of 1.0 mM and expression continued 3 h at 37° C. Cells were harvested by centrifugation at 4K rpm. Cell pellets were resuspended in 100 mL buffer TE (10 mM Tris-HCl, 1 mM EDTA, pH 8.0) and lysis was accomplished using a French Press microfluidizer (two passages). The crude extract, containing BACE1 as insoluble inclusion bodies, was centrifuged at 14K rpm for 15 minutes, and the resulting pellet washed by resuspension in PBS (10 mM sodium phosphate, 150 mM NaCl, pH 7.4) followed by centrifugation at 14K rpm for 20 minutes. Washed inclusion body pellets were solubilized in 50 mM CAPS, 8 M urea, 1 mM EDTA, and 100 mM β-mercaptoethanol, pH 10, and remaining insoluble debris removed by centrifugation at 20K rpm for 30 minutes. BACE1 was refolded by slow injection of the urea-solubilized protein to between 50 and 100 volumes of rapidly stirred water, or 10 mM Na₂CO₃, pH 10, followed by incubation at room temperature for 3-7 days. When BACE1 enzymatic activity no longer increased over time, the pH of the refolding solution was adjusted to 8.0 by addition of 5 mM (final concentration) Tris-HCl, and loaded onto a Q-Sepharose column. Protein was eluted using a linear gradient of 0 to 500 mM NaCl in 10 mM Tris-HCl, pH 8.0. BACE1 was further purified by S-Sepharose chromatography at pH 4.5. Purified enzyme was dialyzed versus 20 mM Tris, 0.125 M NaCl, pH 7.2 at 4° C., and stored at 4° C. Protein concentrations were determined by absorbance at 280 nm, using ε₂₈₀^{1%}=(0.74).

### EXAMPLE 10

### Cloning and Mutagenesis of Mitogen-Activated Protein Kinase/Extracellular Signal-Regulated Kinase Kinase (MEK)

Mek-1 (accession number SWS Q02750) is a dual specificity kinase that plays a key role in cellular proliferation and survival in response to mitogenic stimuli. Mek-1 is the central component of a three-kinase cascade commonly called a MAP kinase cascade. This Raf-Mek-Erk kinase cascade transmits information from cell surface receptors (e.g. EGFR, HER2, PDGFR, FGFR, IGF, etc.) to the nucleus. This pathway is upregulated in approximately 30% of all tumor types, either through overexpression of specific cell surface receptors (e.g. HER2 in breast cancers) or through activating mutations in Ras, a key upstream component of this pathway. Disruption of Mek-1 function has dramatic anti-tumor effects, both in cell culture and in animals. Mek-2 (accession number SWS P36507) is a dual specificity kinase that is both highly homologous (79% identity) to Mek-1 and coordinately expressed with Mek-1. Thus, Mek-1 and Mek-2 represent attractive targets for the development of novel anti-cancer therapeutics. There are no crystal structures to date for Mek-1 or Mek-2.

Cloning of human Mek-1 and Mek-2

Numbering of the wild type and mutant Mek-1 and Mek-2 residues begins at their respective amino termini, with residue number 1 being the initiation methionine, according to the NCBI reported sequences (NCBI accession number L05624 for Mek-1 and NCBI accession number HUMMEK2F for Mek-2). All standard cloning and mutagenesis steps were carried out according to the recommendations of the enzyme manufacturer.

The DNA encoding human Mek-1 was isolated from plasmid pUSE MEK1 (Upstate Biotechnology) and inserted into plasmid pGEX-4T-1 (Amersham) in frame with GST as follows. First, pUSE MEK1 was digested with NotI (New England Biolabs), the 3′ overhang filled in with the Klenow fragment of DNA polymerase (New England Biolabs), and the 1193 bp product encoding MEK1 was isolated from an agarose gel. pGEX-4T-1 was linearized by digestion with EcoRI (New England Biolabs) and the 3′ overhang similarly filled in with the Klenow fragment of DNA polymerase (New England Biolabs). The MEK1 and pGEX-4T-1 DNA fragments were then ligated with T4 ligase and amplified in E. coli strain Top10F′ (Invitrogen) to generate plasmid pGEX-MEK1.

The DNA encoding human Mek-2 was isolated from plasmid pUSE MEK2 (Upstate Biotechnology) and inserted into plasmid pGEX-4T-1 (Amersham) in frame with GST as follows. First, pUSE MEK2 was digested with NotI (New England Biolabs), the 3′ overhang filled in with the Klenow fragment of DNA polymerase (New England Biolabs), and the 1213 bp product encoding MEK2 was isolated from an agarose gel. pGEX-4T-1 was linearized by digestion with EcoRI (New England Biolabs) and the 3′ overhang similarly filled in with the Klenow fragment of DNA polymerase (New England Biolabs). The MEK2 and pGEX-4T-1 DNA fragments were then ligated with T4 ligase and amplified in E. coli strain Top10F′ (Invitrogen) to generate plasmid pGEX-MEK2.

Generation of Mek-1 and Mek-2 Cysteine Mutants

All mutagenesis steps were performed using long range PCR. Reactions contained the parent plasmid (2 ng/μl), sense strand mutant primer (0.5 μM), and antisense strand mutant primer (0.5 μM) that are unique to each reaction. In addition, all reactions contained dNTPs (25 μM) and Pfu polymerase (0.05 Units/μl; Stratagene). Reactions were incubated for one minute at 95° C. followed by 16 cycles of (0.5 minutes at 95° C., 1 minute at 55° C., and 2 minutes at 68° C.) and a final 10 minutes at 68° C. Parent plasmid DNA was then digested with DpnI (New England Biolabs) and the remaining linear PCR product was transformed into E. coli strain Top10F′ (Invitrogen). Mutagenized plasmid DNA, the result of in vivo recombination and subsequent amplification, was purified using QIAquick (Qiagen) columns and verified by sequencing.

First, a 6×HIS epitope tag was introduced into pGEX-MEK1, at the carboxy terminus of MEK1, to generate pGEX-MEK1-HIS using the sense and antisense oligonucleotides MEK1-6HIS-s and MEK1-6HIS-as, resepectively. Similarly, a 6×HIS epitope tag was introduced into pGEX-MEK2, at the carboxy terminus of MEK2, to generate pGEX-MEK2-HIS using the sense and antisense oligonucleotides, MEK2-6HIS-s and MEK2-6HIS-as, resepectively.

| | |
|---|---|
| MEK1-6HIS-s | |
| CACGCTGCCAGCATCGGCGTCGACCCAACCCTGGTT | SEQ ID NO:193 |
| CCGCGTGGATCCCATCACCATCACCATCACTGAGCG | |
| GCCAATTCCCGG | |
| MEK1-6HIS-as | |
| CCGGGAATTGGCCGCTCAGTGATGGTGATGGTGATG | SEQ ID NO:194 |
| GGATCCACGCGGAACCAGGGTTGGGTCGACGCCGAT | |
| GCTGGCAGCGTG | |
| MEK2-6HIS-s | |
| ACGCGTACTGCAGTGGGCGTCGACCCAACCCTGGTT | SEQ ID NO:195 |
| CCGCGTGGATCCCATCACCATCACCATCACTGAGCG | |
| GCCAATTCCCGG | |
| MEK2-6HIS-as | |
| CCGGGAATTGGCCGCTCAGTGATGGTGATGGTGATG | SEQ ID NO:196 |
| GGATCCACGCGGAACCAGGGTTGGGTCGACGCCCAC | |
| TGCAGTACGCGT | |

Subsequently, 16 individual mutations were introduced into pGEX-MEK1-HIS. Similarly, the analogous 16 individual mutations were introduced into pGEX-MEK2-HIS. Each of these mutations introduces a cysteine into the MEK1 or MEK2 protein, and each is named according to the resultant amino acid substitution. For example, primer pair MEK1-N78C-sense and MEK1-N78C-antisense were used to introduce a cysteine in place of N78 of MEK1, generating pGEX-MEK1/N78C-HIS.

Mutagenic Oligonucleotides

| | | |
|---|---|---|
| MEK1-N78C-s | GAGCTGGGGGCTGGCTGCGGCGGTGTGGTGTTC | SEQ ID NO:197 |
| MEK1-N78C-as | GAACACCACACCGCCGCAGCCAGCCCCCAGCTC | SEQ ID NO:198 |
| MEK1-G79C-s | CTGGGGGCTGGCAATTGCGGTGTGGTGTTCAAG | SEQ ID NO:199 |
| MEK1-G79C-as | CTTGAACACCACACCGCAATTGCCAGCCCCCAG | SEQ ID NO:200 |
| MEK1-I107C-s | GAGATCAAACCCGCATGCCGGAACCAGATCATA | SEQ ID NO:201 |
| MEK1-I107C-as | TATGATCTGGTTCCGGCATGCGGGTTTGATCTC | SEQ ID NO:202 |
| MEK1-R108C-s | ATCAAACCCGCAATCTGCAACCAGATCATAAGG | SEQ ID NO:203 |
| MEK1-R108C-as | CCTTATGATCTGGTTGCAGATTGCGGGTTTGAT | SEQ ID NO:204 |
| MEK1-I111C-s | GCAATCCGGAACCAGTGCATAAGGGAGCTGCAG | SEQ ID NO:205 |
| MEK1-I111C-as | CTGCAGCTCCCTTATGCACTGGTTCCGGATTGC | SEQ ID NO:206 |
| MEK1-E114C-s | AACCAGATCATAAGGTGCCTGCAGGTTCTGCAT | SEQ ID NO:207 |
| MEK1-E114C-as | ATGCAGAACCTGCAGGCACCTTATGATCTGGTT | SEQ ID NO:208 |
| MEK1-L118C-s | AGGGAGCTGCAGGTTTGCCATGAGTGCAACTCT | SEQ ID NO:209 |
| MEK1-L118C-as | AGAGTTGCACTCATGGCAAACCTGCAGCTCCCT | SEQ ID NO:210 |
| MEK1-V127C-s | AACTCTCCGTACATCTGCGGCTTCTATGGTGCG | SEQ ID NO:211 |
| MEK1-V127C-as | CGCACCATAGAAGCCGCAGATGTACGGAGAGTT | SEQ ID NO:212 |
| MEK1-M143C-s | GAGATCAGTATCTGCTGCGAGCACATGGATGGA | SEQ ID NO:213 |
| MEK1-M143C-as | TCCATCCATGTGCTCGCAGCAGATACTGATCTC | SEQ ID NO:214 |
| MEK1-S150C-s | CACATGGATGGAGGTTGCCTGGATCAAGTCCTG | SEQ ID NO:215 |
| MEK1-S150C-as | CAGGACTTGATCCAGGCAACCTCCATCCATGTG | SEQ ID NO:216 |
| MEK1-L180C-s | AAAGGCCTGACATATTGCAGGGAGAAGCACAAG | SEQ ID NO:217 |
| MEK1-L180C-as | CTTGTGCTTCTCCCTGCAATATGTCAGGCCTTT | SEQ ID NO:218 |
| MEK1-I186C-s | AGGGAGAAGCACAAGTGCATGCACAGAGATGTC | SEQ ID NO:219 |
| MEK1-I186C-as | GACATCTCTGTGCATGCACTTGTGCTTCTCCCT | SEQ ID NO:220 |
| MEK1-K192C-s | ATGCACAGAGATGTCTGCCCCTCCAACATCCTA | SEQ ID NO:221 |
| MEK1-K192C-as | TAGGATGTTGGAGGGGCAGACATCTCTGTGCAT | SEQ ID NO:222 |
| MEK1-S194C-s | AGAGATGTCAAGCCCTGCAACATCCTAGTCAAC | SEQ ID NO:223 |
| MEK1-S194C-as | GTTGACTAGGATGTTGCAGGGCTTGACATCTCT | SEQ ID NO:224 |
| MEK1-L197C-s | AAGCCCTCCAACATCTGCGTCAACTCCCGTGGG | SEQ ID NO:225 |
| MEK1-L197C-as | CCCACGGGAGTTGACGCAGATGTTGGAGGGCTT | SEQ ID NO:226 |
| MEK1-V211C-s | CTCTGTGACTTTGGGTGCAGCGGGCAGCTCATC | SEQ ID NO:227 |
| MEK1-V211C-as | GATGAGCTGCCCGCTGCACCCAAAGTCACAGAG | SEQ ID NO:228 |
| MEK2-N82C-s | GAGCTGGGCGCGGGCTGCGGCGGGGTGGTCACC | SEQ ID NO:229 |
| MEK2-N82C-as | GGTGACCACCCCGCCGCAGCCCGCGCCCAGCTC | SEQ ID NO:230 |
| MEK2-G83C-s | CTGGGCGCGGGCAACTGCGGGGTGGTCACCAAA | SEQ ID NO:231 |
| MEK2-G83C-as | TTTGGTGACCACCCCGCAGTTGCCCGCGCCCAG | SEQ ID NO:232 |
| MEK2-I111C-s | GAGATCAAGCCGGCCTGCCGGAACCAGATCATC | SEQ ID NO:233 |
| MEK2-I111C-as | GATGATCTGGTTCCGGCAGGCCGGCTTGATCTC | SEQ ID NO:234 |
| MEK2-R112C-s | ATCAAGCCGGCCATCTGCAACCAGATCATCCGC | SEQ ID NO:235 |
| MEK2-R112C-as | GCGGATGATCTGGTTGCAGATGGCCGGCTTGAT | SEQ ID NO:236 |
| MEK2-I115C-s | GCCATCCGGAACCAGTGCATCCGCGAGCTGCAG | SEQ ID NO:237 |
| MEK2-I115C-as | CTGCAGCTCGCGGATGCACTGGTTCCGGATGGC | SEQ ID NO:238 |
| MEK2-E118C-s | AACCAGATCATCCGCTGCCTGCAGGTCCTGCAC | SEQ ID NO:239 |
| MEK2-E118C-as | GTGCAGGACCTGCAGGCAGCGGATGATCTGGTT | SEQ ID NO:240 |
| MEK2-L122C-s | CGCGAGCTGCAGGTCTGCCACGAATGCAACTCG | SEQ ID NO:241 |
| MEK2-L122C-as | CGAGTTGCATTCGTGGCAGACCTGCAGCTCGCG | SEQ ID NO:242 |
| MEK2-V131C-s | AACTCGCCGTACATCTGCGGCTTCTACGGGGCC | SEQ ID NO:243 |
| MEK2-V131C-as | GGCCCCGTAGAAGCCGCAGATGTACGGCGAGTT | SEQ ID NO:244 |
| MEK2-M147C-s | GAGATCAGCATTTGCTGCGAACACATGGACGGC | SEQ ID NO:245 |
| MEK2-M147C-as | GCCGTCCATGTGTTCGCAGCAAATGCTGATCTC | SEQ ID NO:246 |
| MEK2-S154C-s | CACATGGACGGCGGCTGCCTGGACCAGGTGCTG | SEQ ID NO:247 |
| MEK2-S154C-as | CAGCACCTGGTCCAGGCAGCCGCCGTCCATGTG | SEQ ID NO:248 |
| MEK2-L184C-s | CGGGGCTTGGCGTACTGCCGAGAGAAGCACCAG | SEQ ID NO:249 |
| MEK2-L184C-as | CTGGTGCTTCTCTCGGCAGTACGCCAAGCCCCG | SEQ ID NO:250 |
| MEK2-I190C-s | CGAGAGAAGCACCAGTGCATGCACCGAGATGTG | SEQ ID NO:251 |
| MEK2-I190C-as | CACATCTCGGTGCATGCACTGGTGCTTCTCTCG | SEQ ID NO:252 |
| MEK2-K196C-s | ATGCACCGAGATGTGTGCCCCTCCAACATCCTC | SEQ ID NO:253 |
| MEK2-K196C-as | GAGGATGTTGGAGGGGCACACATCTCGGTGCAT | SEQ ID NO:254 |
| MEK2-S198C-s | CGAGATGTGAAGCCCTGCAACATCCTCGTGAAC | SEQ ID NO:255 |
| MEK2-S198C-as | GTTCACGAGGATGTTGCAGGGCTTCACATCTCG | SEQ ID NO:256 |
| MEK2-L201C-s | AAGCCCTCCAACATCTGCGTGAACTCTAGAGGG | SEQ ID NO:257 |
| MEK2-L201C-as | CCCTCTAGAGTTCACGCAGATGTTGGAGGGCTT | SEQ ID NO:258 |
| MEK2-V215C-s | CTGTGTGACTTCGGGTGCAGCGGCCAGCTCATA | SEQ ID NO:259 |
| MEK2-V215C-as | TATGAGCTGGCCGCTGCACCCGAAGTCACACAG | SEQ ID NO:260 |

Sequencing Primers

| | | |
|---|---|---|
| pGEX forward | GGGCTGGCAAGCCACGTTTGGTG | SEQ ID NO:261 |
| pGEX reverse | CCGGGAGCTGCATGTGTCAGAGG | SEQ ID NO:262 |

Expression of Mek-1 and Mek-2 Mutants

Mutant alleles of Mek-1 and Mek-2 were expressed in E. coli and purified essentially as described for Mek-1 [by McDonald, O. B., et al., Analytical Biochem. 268: 318-329 (1999)]. Plasmids containing the mutant Mek-1 and Mek-2 alleles were transformed into BL21 DE3 pLysS cells (Invitrogen) according to manufacturer's suggestions. Cultures were grown overnight at 37° C. from single colonies in 100 ml 2YT medium supplemented with 100 μg/ml ampicillin and 100 μg/ml chloramphenicol. This culture was then added to 1.5 L 2YT supplemented with 100 μg/ml ampicillin to achieve an OD₆₀₀ of approximately 0.05 and then grown to an OD₆₀₀ of approximately 0.7 at 30° C. Expression was induced with the addition of IPTG to a final concentration of 1 mM and the culture was incubated for four hours at 25° C. Cells were pelleted in a Sorfall GSA rotor at 6K rpm for 15 minutes and stored at −80° C.

Mek-1 and Mek-2 mutants were purified from cells by first resuspending cell pellets in ice cold PBS containing 0.5% Triton X-100 and incubating on ice for 45 minutes, followed by extensive sonication. Lysates were clarified by centrifugation in a Sorvall GSA rotor at 12K rpm for one hour. Fusion proteins were first purified on Ni-NTA resin (Qiagen) according to manufacturer's suggestions, followed by further purification on glutathione agarose as described [by McDonald, O. B., et al., Analytical Biochem. 268: 318-329 (1999)]. Epitope tags were removed with thrombin cleavage and aliquots of purified protein were stored at −80° C. in TBS containing 10% glycerol.

### EXAMPLE 11

### Cloning and Mutagenesis of Human Cathepsin S (CATS)

Cathepsin S (accession number SWS P25774) is a thiol protease located primarily in the lysosome. This enzyme plays roles in antigen presentation by processing of the MHC-II antigen receptor; thus inhibitors to the enzyme could be used for diseases such as inflammation and autoimmunity such as rheumatoid arthritis, multiple sclerosis, asthma and organ rejection. It has also been reported that catS is present in increased levels in the Alzheimer's disease and Down Syndrome brain compared with normal brain. A structural model of cathepsin S [1BXF, Fengler, A. & Brandt W., Protein Eng 11:1007-1013(1998)] and a crystal structure of the C25S mutant [Turkenburg, J. P. et al. Acta Crystallogr D Biol Crystallog 58: 451-455 (2002)] are available.

Cloning of Human catS

The DNA sequence encoding human cathepsin S (catS) was isolated by PCR from the plasmid pDualGC (Stratagene #E01089) using PCR primers listed below corresponding to the protein N- and C-termini. These primers were designed to contain restriction endonuclease sites EcoRI and XhoI, for subcloning into a modified pFastBac vector, pFBHT (c.f. example 4 above). SEQ ID NO: 263 was used with SEQ ID NO: 264 and SEQ ID NO 265 to make catS with and without a 6×his tag, respectively.

| | |
|---|---|
| 5′CatS EcoRI | |
| CCGGAATTCATGAAACGGCTGGTTTGTGTGCT | SEQ ID NO:263 |
| 3′CatS XhoI | |
| CCCCGCTCGAGGATTTCTGGGTAAGAGGGAAAG | SEQ ID NO:264 |
| 3′CatS XhoI stop | |
| CCCCGCTCGAGCTAGATTTCTGGGTAAGAGGGAAA | SEQ ID NO:265 |

The PCR reaction was purified on a Qiaquick PCR purification column (Qiagen). The PCR product containing the catS sequence was cut with restriction endonucleases (42 μl PCR product, 1 μl each endonuclease, 5 μl appropriate 10×buffer; incubated at 37° C. for 3 hours). The pFBHT vector was cut with restriction endonucleases (5 μg DNA, 1 μl each endonuclease, 3 μl appropriate 10×buffer, water to 30 μl; incubated at 37° C. for 3 hours; added 1 μl CIP and incubated at 37° C. for 60 minutes). The products of nuclease cleavage were isolated from an agarose gel (1% agarose, TBE buffer) and ligated together using T4 DNA ligase (50 ng pFBHT vector and 50 ng catS PCR product in 10 μl, 10 μl 2×ligase buffer (Roche), 1 μl ligase, incubated at 25° C. for 15 minutes). 1 μl of the ligation reaction was transformed into Library Efficiency Chemically Competent DH5α cells (Invitrogen) (1 μl ligation reaction, 100 μl competent cells; incubated at 4° C. for 30 minutes, 42° C. for 45 seconds, 4° C. for 2 minutes, then 900 μl SOC media was added and incubated for 1 hour with shaking at 225 rpm at 37° C.), and plated onto LB/agar plates containing 100 μg/ml ampicillin. After incubation at 37° C. overnight, single colonies were grown in 3 ml LB media containing 100 μg/ml ampicillin for 8 hours. Cells were then isolated and double-stranded DNA extracted from the cells using a Qiagen DNA miniprep kit. Sequencing of catS gene was accomplished using M13/pUC Forward and Reverse Amplification Primers (Invitrogen #18430-017).

Generation of CatS Cysteine Mutations

Mutations were generated using as previously described [Kunkel T. A., et al., Methods_{—}Enzymol. 154: 367-382 (1987)]. DNA oligonucleotides used are shown below and were designed to hybridize with sense strand DNA from plasmid. Sequences were verified using primers with SEQ ID NO: 74 and SEQ ID NO: 75.

Mutagenic Oligonucleotides

| | | |
|---|---|---|
| Y18C | CACAAGAACCTTGACATTTCACTTCAGT | SEQ ID NO:266 |
| K64C | CACCATTGCAGCCACAGTTTCCATATTT | SEQ ID NO:267 |
| N67C | CATGAAGCCACCACAGCAGCCTTTGTT | SEQ ID NO:268 |
| T72C | CTGGAAAGCCGTGCACATGAAGCCACC | SEQ ID NO:269 |
| E115C | GCCATAAGGAAGGCAAGTGTACTTTGA | SEQ ID NO:270 |
| R141C | GAAAGAAGGATGACACGCATCTACACC | SEQ ID NO:271 |
| F146C | ACTTCTGTAGAGGCAGAAAGAAGGATG | SEQ ID NO:272 |
| F211C | TGGGTAAGAGGGACAGCTAGCAATCCC | SEQ ID NO:273 |

Scrub mutations of the cysteines were also made using the following oligonucleotides.

| | | |
|---|---|---|
| C12A | CACTTCAGTAACAGCCCCTTTCTCTCTC | SEQ ID NO:274 |
| C12Y | CACTTCAGTAACATACCCTTTCTCTCTC | SEQ ID NO:275 |
| C25S | CACTGAAAGCCCAGGAAGCACCACAAGA | SEQ ID NO:276 |
| C110A | CAGTGTACTTTGAAGCTGTGGCAGCACG | SEQ ID NO:277 |

Expression of CatS Mutant Proteins

All CatS-FBHT plasmids were site-specifically transposed into the baculovirus shuttle vector (bacmid) by transforming the plasmids into DH10bac (Gibco/BRL) competent cells as follows: 1 μl DNA at 5 ng/μl, 10μl 5×KCM [0.5 M KCl, 0.15 M CaCl₂, 0.25 M MgCl₂], 30 μl water was mixed with 50 μl PEG-DMSO competent cells, incubated at 4° C. for 20 minutes, 25° C. for 10 minutes, added 900 μl SOC and incubated at 37° C. with shaking for 4 hours, then plated onto LB/agar plates containing 50 μg/ml kanamycin, 7 μg/ml gentamycin, 10 μg/ml tetracycline, 100 μg/ml Bluo-gal, 10 μg/ml IPTG. After incubation at 37° C. for 24 hours, large white colonies were picked and grown in 3 ml 2YT media overnight. Cells were then isolated and double-stranded DNA was extracted from the cells as follows: pellet was resuspended in 250 μl of Solution 1 [15 mM Tris-HCl (pH 8.0), 10 mM EDTA, 100 μg/ml RNase A]. Added 250 μl of Solution 2 [0.2 N NaOH, 1% SDS] mixed gently and incubated at room temperature for 5 minutes. Added 250 μl 3 M potassium acetate, mixed and placed on ice for 10 minutes. Centrifuged 10 minutes at 14,000×g and transferred supernatant to a tube containing 0.8 ml isopropanol. Mix and place on ice for 10 minutes. Centrifuge 15 minutes at 14,000×g, wash with 70% ethanol, air dry pellet and resuspended DNA in 40 μl TE.

The bacmid DNA was used to transfect Sf9 cells. Sf9 cells were seeded at 9×10⁵ cells per 35 mm well in 2 ml of Sf-900 II SFM medium containing 0.5×concentration of antibiotic-antimycotic and allowed to attach at 27° C. for 1 hour. During this time, 5 μl of bacmid DNA was diluted into 100 μl of medium without antibiotics, 6 μl of CellFECTIN reagent was diluted into 100 μl of medium without antibiotics and then the 2 solutions were mixed gently and allowed to incubate for 30 minutes at room temperature. The cells were washed once with medium without antibiotics, the medium was aspirated and then 0.8 ml of medium was added to the lipid-DNA complex and overlaid onto the cells. The cells were incubated for 5 hours at 27° C., the transfection medium was removed and 2 ml of medium with antibiotics was added. The cells were incubated for 72 hours at 27° C. and the virus was harvested from the cell culture medium.

The virus was amplified by adding 1.0 ml of virus to a 50 ml culture of Sf9 cells at 2×10⁶ cells/ml and incubating at 27° C. for 72 hours. The virus was harvested from the cell culture medium and this stock was used to express the various catS constructs in High-Five cells. A 1 L culture of High-Five cells at 2×10⁶ cells/ml was infected with virus at an approximate MOI of 2 and incubated for 72 hours. Cells were pelleted by centrifugation and the supernatant was dialyzed against 20 L Load buffer (50 mM NaH₂PO₄, pH 8.0, 300 mM NaCl, 10 mM imidazole), filtered and loaded onto a Ni-NTA (Superflow Ni-NTA, Qiagen) column at 1 ml/min, washed with Load buffer at 2 ml/min and eluted with 50 mM NaH₂PO₄, pH 8.0, 300 mM NaCl, 250 mM imidazole.

### EXAMPLE 12

### Caspase-1

Caspase-1 (accession number SWS P25774), like other caspases exists as an inactive proform, and is proteolytically processed into a large subunit and a small subunit, which then combine to form the active enzyme. An important substrate of caspase-1 is the proform of interleukin-1 (beta). Caspase-1 produces the active form of this cytokine, which plays a role in processes such as inflammation, septic shock and wound healing. Additionally, active capase-1 induces apoptosis, and plays a role in the progression Huntington's disease. The structure of caspase-1 has been solved [1BMQ, Okamoto, Y., et al., Chem Pharm Bull (Tokyo), 47:11-21 (1999)].

### IL-13

IL-13 (accession number SWS P35225), which is produced mainly by activated Th2 cells, shows structural and functional similarities to IL-4. Like IL-4, it increases the secretion of immunoglobulin E by B cells and is involved in the expulsion of parasites. In addition, IL-13 downregulates the production of cytokines including IL-1b, IL-6, TNF-alpha and IL-8 by stimulated monocytes. IL-13 also prolongs monocyte survival, increases the expression of MHC class II and CD23 on the surface of monocytes, and increases expression of CD23 on B cells. Furthermore, IL-2 and IL-13 synergize in the regulation interferon-gamma synthesis. Due to these effects, IL-13 plays a role in conditions such as allergy and asthma. In particular, a polymorphism at position 130 (Q) increases the risk of asthma development. The structure of IL-13 has been solved by nuclear magnetic resonance (NMR) [1GA3, Eissenmesser, E. Z. et al., J. Mol. Biol. 310: 231-241 (2001)].

### CD40L

CD40L (accession number SWS P29965) is a protein that is found in two forms, a transmembrane form and also an active, proteolytically processed, extracellular soluble form. The transmembrane form is expressed on the surface of CD4+ T lymphocytes. Like other members of the TNF family, it is forms a homotrimer. CD40L mediates the proliferation of B cells, epithelial cells, fibroblasts, and smooth muscle cells. Binding of CD40L to the CD40 receptor on T cells provides a critical signal for isotype class switching and production of immunoglobulin antibodies. Defects in CD40L lead to an elevation in IgM levels, and an deficiency in all other immunoglobulin subtypes. Inhibitors to CD40L would find use in the treatment of autoimmune disease and graft rejection. In addition, reduced interaction between CD40L and its receptor reduces the degree of tau hyperphosphorylation in a mouse model of Alzheimer's disease. The crystal structure of CD40L has been solved [1ALY, Karpusas, M., et al., Structure 3:1031-1039(1995), erratum in Structure 3:1046 (1995)].

### Human B-Cell Activating Factor (BAFF)

A member of the TNF superfamily, BAFF (accession number SWS Q9Y275) is a homotrimer and found in both transmembrane and soluble forms. The transmembrane form is processed by the furin family of proprotein convertases. BAFF is upregulated by interferon-gamma and downregulated by PMA/ionomycin treatment. BAFF binds to three different receptors. When it binds to the B-cell specific receptor (BAFFR), it promotes survival of B-cells and the B-cell response. Furthermore, both BAFF and a proliferation-inducing ligand (APRIL) bind to the receptors transmembrane activator and CAML interactor (TACI) and B cell maturation antigen (BCMA), forming a 2 ligands-2 receptors pathway that is responsible for stimulation of T-cell and B-cell function and humoral immunity. Inhibitors of BAFF would serve as therapeutics for autoimmune diseases characterized by abnormal B-cell activity, such as systemic lupus erythematosis (SLE) and rheumatoid arthritis (RA). A structure of the soluble protein is available [1JH5, Liu, Y., et al., Cell, 108: 383-394 (2002)].

### Tumor Suppressor P53

P53 (accession number SWS P04637), a transcription factor that can suppress tumor growth, binds DNA as a homotetramer and is activated by phosphorylation of a serine residue. There are two mechanisms of tumor suppression, depending upon the cell type: induction of growth arrest and activation of apoptosis. P53 controls cell growth by regulating expression of a set of genes; for example, it increases the transcription of an inhibitor of cyclin-dependent kinases. Apoptosis results from the p53-mediated stimulation of Bax or Fas expression, or the decrease in Bc12 expression. P53 is mutated or inactivated in about 60% of known cancers, and is also often overexpressed in a variety of tumor tissues. Reversible inhibitors of p53 could be used as an adjunct to conventional radio- and chemotherapy to prevent damage to normal tissues during treatment and its severe side effects. Such an inhibitor was shown to protect mice from lethal doses of radiation without the promotion of tumor formation. There is a crystal structure of human p53 bound to Xenopus laevis mdm2 protein [1YCQ, Kussie, P. H., et al., Science 274: 948-953 (1996)].

### P53-Binding Protein MDM2

In response to DNA damage, p53 increases the transcription of the protein mdm2 (accession number SWS Q00987). In a form of negative feedback, mdm2 inhibits p53-induced cell cycle arrest and apoptosis by two means. Firstly, mdm2 binds the transcriptional activation domain of p53, reducing its transcriptional activation activity. Secondly, in the presence of ubiquitin E1 and E2, mdm2 serves as an ubiquitin protein ligase E3 for both itself and p53. The ubiquitination of p53 allows its export from the nucleus to the proteasome, where it is destroyed. There are eight isoforms of mdm2 that are produced by alternative splicing. They are mdm2, mdm2-A, mdm2-A1, mdm2-B, mdm2-C, mdm2-D, mdm2-E, and mdm2-alpha. Of these, mdm2-A, mdm2-B, mdm2-C, mdm2-D, and mdm2-E are observed in human cancers but not in normal tissues. Mdm2 amplification has also been observed in certain tumor types, including soft tissue sarcoma, osteosarcoma, and glioblastoma. These tumors often contain wild type p53. Small molecule inhibitors of mdm2 could promote the proapoptotic activity of the wild type p53 and find use in cancer therapy. The structure of Xenopus laevis mdm2 in complex with human p53 has been solved [1YCR, Kussie, P. H. et al., Science 274: 948-953 (1996)].

### Bcl-x

Bcl-x (accession number SWS Q07817) is a member of the Bcl2 family of proteins and has two major isoforms produced by alternative splicing, bcl-x(L), bcl-x(S). The long isoform, bcl-x(L) is found in long-lived postmitotic cells and inhibits apoptosis, whereas the short isoform, bcl-x(S), is found in cells with a high turnover rate and promotes apoptosis. The long isoform inhibits apoptosis by binding to voltage-dependent anion channel (VDAC) and preventing the release of apopotosis activator cytochrome c from the mitochondrial membrane. This antiapoptotic activity is dependent upon the BH4 (bcl-2 homology) domain of Bcl-x(L); binding of this protein to other Bcl2 family members is dependent upon the BH1 and BH2 domains. Expression of Bcl-x(L) has been observed to be expressed primarily by the neoplastic cells in a majority of lymphoma cases. Inhibition of bcl-x(L) expression in several cell lines resulted in apoptosis. Thus, due to its antiapoptotic effects, bcl-x(L) is a target for cancer therapeutics. Interestingly, binding of Bcl-x(L) to another Bcl2 family member, the proapoptotic protein Bax, results in an increase in apoptosis (see below). A crystal structure of Bcl-x(L) has been solved [1MAZ, Muchmore, S. W., et al. Nature 381: 335-341 (1996)].

### Bax

Bax [accession number SWS Q07812 (BAX alpha); SWS Q07814 (BAX beta); SWS Q07815 (BAX gamma); SWS P55269 (BAX delta)] promotes apoptosis by binding to the antiapoptotic protein bcl-x(L), inducing the release of cytochrome c, and activating caspase-3. Bax has several isoforms produced by alternative splicing; some are membrane bound and others are cytoplasmic. The BH3 domain of Bax is necessary for its binding to members of the anti-apoptotic Bcl2 family. Defects in Bax are observed in some cell lineages from hematopoietic cancers. Bax agonists could be used in cancer therapies, while Bax inhibitors could be used to counteract neuronal cell death resulting from ischemia, spinal cord injury, Parkinson's disease and Alzheimer's disease. An NMR structure of BAX has been solved [1F16, Suzuki, M., et al., Cell 103:645-654 (2000)].

### CDC25A

CDC25A (accession number SWS P30304) is a dual-specificity phosphatase also known as M-phase inducer phosphatase 1 (MPI1). Induced by cyclin B, CDC25A is required for progression of the cell cycle, and induces mitosis in a dosage-dependent manner. CDC25 directly dephosphorylates CDC2, thereby decreasing its activity. It has also been demonstrated in vitro that CDC25 dephosphorylates CDK2 in complex with cyclinE. Elevated levels of CDC25 can trigger uncontrolled cell growth and are linked with increased mortality in breast cancer patients. Activated CDC25A is also observed in degenerating neurons of the Alzheimer's diseased brain. A structure of the catalytic core has been solved [1C25, Fauman, F. B., et al., Cell 93: 617-625 (1998)].

### CD28

CD28 (accession number SWS P10747) is a disulfide-linked homodimenic transmembrane protein expressed on activated B-cells and a subset of T-cells. This protein can bind three others: B7-1, B7-2, and CTLA-4. The interaction of CD28 with B7-1 and B7-2 present on the surface of antigen presenting cells (APCs) results in a co-stimulation of naïve T-cell activation, whereas subsequent interaction of the same B7-1 and B7-2 molecules with CTLA-4 leads to an attenuation of the T-cell stimulation. CD28-associated signaling pathways are important therapeutic targets for autoimmune disease, graft vs. host disease (GVHD), graft rejection, and promotion of immunity against tumors. The structure of CD28 has not been solved to date.

### B7

There are 2 B7 proteins: B7-1 (accession number SWS P33681), also known as CD80, and B7-2 (accession number SWS P42081), also known as CD86. Both are highly glycosylated transmembrane proteins expressed on activated B-cells. Early events in immune response are controlled by the interactions of these molecules with CD28 and CTLA-4 (see above). Thus B7-1 and B7-2 make significant targets for therapeutics treating autoimmune disease. A structure of the soluble form of B7-1 has been solved [1DR9, Ikemizu, S., et al., Immunity 12: 51-60 (2000)] in addition to a structure of B7-1 in complex with CTLA-4 [1I8L, Stamper, C. C., et al., Nature 410: 608-611 (2001)]. In addition, a structure of B7-2 in complex with CTLA-4 has been solved [1I85, Schwartz, J. -C. D., et al., Nature 410: 604-608 (2001)].

### C5A

The immune system comprises in part the complement cascade, which is a set of more than 20 proteins. C5a is one of these complement proteins; it is a cytokine-like activation product of C5. C5a effects inflammation, and specifically has a role in the recruitment of neutrophils in response to bacterial infection. In sepsis, the life threatening spread of bacterial toxins through the blood, the effects of C5a are exhausted, due to an overexposure of the neutrophils to excessive amounts of this complement protein. Furthermore, expression levels of C5a receptor (accession number SWS P21730) are increased in certain vital organs during sepsis. Thus inhibitors of C5a or the C5a receptor could help in treating sepsis. Inhibitors of C5a could also be used in the treatment of bullous pemphigoid, the most common autoimmune blistering disease. Another effect of C5a is its synergy with the Abeta peptide to promote secretion of IL-1 and IL-6 in human macrophage-like THP-1 cells; C5a may therefore be involved in the pathogenesis of Alzheimer's disease. Although the structure of C5a has been solved by NMR [1KJS, Zhang, X, et al., Proteins 28: 261-267 (1997)], there is no structure of the C5a receptor to date.

### AKT

Akt is an important component of the signaling pathway of growth factor receptors. There are three highly related Akt genes, Akt 1-3 (accession numbers SWS P31749, Akt1; SWS P31751, Akt2; SWS Q9Y243), which show compensatory effects for one another. However, they have different expression patterns, suggesting that each may have unique functions as well. Each Akt is activated by phosphorylation of multiple residues and is activated by the kinase ILK. Binding of activated Akt to P13K (phosphatidyl inositol 3-kinase) causes the translocation of the active Akt to the plasma membrane. Akt has pleiotropic effects leading to cell survival. Additionally, Akt amplification and elevated levels of Akt have been found in some types of cancers. A crystal structure of the kinase domain of Akt2, also known as PKB-β, has recently been obtained [Yang, J., et al., Molecular Cell 9: 1227-1240 (2002)].”

### CD45

CD45 (accession number SWS P08575) is a receptor protein tyrosine phosphatase that is primarily located in the plasma membrane of leukocytes; it has several isoforms differing in the extracellular domain, the significance of which is presently unknown. Substrates for CD45 include the kinases lyc, fyn, and other src kinases. Additionally, CD45 engages in noncovalent interactions with the lymphocyte phosphatase associated protein (LPAP). CD45 is critical for activation through the antigen receptor on T cells and B cells, and may also be important for the antigen-mediated activation in other leukocytes. Dimerization of CD45 disables its function. Inhibitors of CD45 could be used to prevent allograft rejection. There is no structure of CD45 to date.

### Tyrosine Kinase-Type Cell Surface Receptor HER2

HER-2 (accession number SWS P04626), otherwise known as ErbB2 is a receptor tyrosine kinase that is related to EGFR (ErbB1). Although there are no known ligands for HER-2 in isolation, when HER-2 dimerizes with other members of the ErbB family, i.e., ErbB1, ErbB3 and ErbB4, the dimeric complex can bind to a number of ligands. These ligands include heregulins, EGF, betacellulin, and NRG, although binding depends upon which ErbB proteins are in the heterodimer. Ligand binding increases the phosphorylation of HER-2, and effects subsequent intracellular signaling steps. HER-2 is frequently overexpressed in breast cancer cells, and this overexpression may mediate their proliferation. Breast cancer cells overexpressing HER-2 are also more responsive to HER-2 inhibitors. HER-2 is also implicated in a number of other cancers, such as ovarian, prostate, lung, fallopian tube, osteosarcoma, and childhood medulloblastoma. The structure of this receptor has not yet been solved.

### Human Glycogen Synthase Kinase-3 (GSK-3)

GSK-3 (accession numbers SWS P49840, GSK-3α; SWS P49841, GSK-3β) is involved in the hormonal control of Myb, glycogen synthase, and c-jun. The phosphorylation of c-jun by GSK-3 decreases the affinity of c-jun for DNA. Additionally, GSK-3 is phosphorylated by ILK-1 and Akt-1. Phosphorylation by Akt1 causes the inhibition of catalytic activity of GSK-3, which normally phosphorylates cyclin D, thereby targeting cyclin D for destruction. The net effect of this phosphorylation of GSK-3 is the promotion of cell survival. Increased GSK-3 activity has been found in tissue from diabetic patients, consistent with its role in the development of insulin resistance. Furthermore, GSK-3β is overexpressed in the Alzheimer's disease brain, and this overexpression is associated with tau protein hyperphosphorylation, a hallmark of the disease. Finally, the effects of some mood-stabilizing drugs such as lithium appear to be mediated by inhibition of GSK. Therefore it is possible that GSK-3 inhibitors would increase the effectiveness of some psychoactive drugs. There is a structure available for GSK-3β [1H8F, Dajani, R., et al., Cell 105: 721-732 (2001)].

### Alpha-E/Beta-7

The protein complex alpha-E/beta-7 is a transmembrane integrin that plays a role in lymphocyte migration and homing. Specifically, the complex serves as a receptor for E-cadherin. Alpha-E (accession number SWS P38570) is made up of two subunits, α and β, the α-subunit itself is composed of a light chain and a heavy chain linked by a disulfide bond. Likewise, beta-7 (accession number SWS P26010) is also composed of α- and β-subunits. The alpha-E/beta-7 complex normally mediates the adhesion of intra-epithelial T lymphocytes to mucosal epithelial cell layers; it also plays a role in the dissemination of non-Hodgkin's lymphoma. Furthermore, a possible mechanism of inflammation involves migration of lymphocytes from the gut epithelium to other parts of the body. Changes in alpha-E/beta-7 levels have been observed in a variety of diseases. Elevated levels of this integrin have been observed in patients with Systemic Lupus Erythematosus (SLE), in the lung epithelium of patients with interstitial lung disease, and in the sinovial fluid of patients with rheumatoid arthritis. Altered patterns of alpha-E/beta-7 expression have been observed in patients with Crohn's disease, and antibodies to this complex were shown to prevent immunization-induced colitis in a mouse model. Hence, inhibitors to this complex would be valuable in the treatment of inflammation, especially mucosal inflammation. There are no structures available for alpha-E or beta-7.

### Tissue Factor

Human tissue factor (accession number SWS P13726), also known as thromboplastin, is an integral transmembrane protein that is normally located at the extravascular cell surface. Upon injury to the skin, tissue factor is exposed to blood and complexes with the active form of coagulation enzyme Factor VII, known as Factor VIIA (see below). Tissue factor can bind both the inactive and active forms of coagulation Factor VII, and is an obligate cofactor for Factor VIIA in triggering the coagulation cascade. Furthermore, since Tissue Factor plays a major role in thrombosis, inhibition of this factor would be expected to decrease the risk for clinical outcomes of thrombosis such as atherosclerosis, arterial occlusion, stroke, and myocardial infarction. A structure of the extracellular domain of tissue factor has been solved [2HFT, Muller, Y. A., et al., J. Mol Biol 256:144-159 (1996)].

### Factor VII

Factor VII (accession number SWS P08709) is the zymogen (inactive precursor) form of the serine protease coagulation Factor VIIa. More than 99% of this protease circulates in the inactive single-chain form; upon cleavage of an Arg-Ile peptide bond by one of several factors, the active two-chain form is produced. This two-chain form comprises a heavy chain and a light chain, linked by a disulfide bond. Enzymatic carboxylation of Glu residues in Factor VII, which is dependent upon vitamin K, allows the protein to bind calcium. In the presence of calcium and the cofactor human tissue factor (see above), Factor VIIa cleaves Factor X and Factor IX to produce their respective active forms, which propagate the coagulation cascade. Defects in Factor VII can lead to bleeding disorders, where recombinant Factor VIIa finds use as a treatment. Conversely, some polymorphisms of the Factor VII gene have been associated with an increased risk for myocardial infarction, which is often caused by blood clots. Factor VII inhibitors are expected to find use in preventing heart disease. A structure of the zymogen form of factor VII in complex with an inhibitory peptide has been solved [1JBU, Eigenbrot, C., et al., Structure 9:627-636 (2001)].

All references cited throughout the specification are expressly incorporated herein by reference. While the present invention has been described with reference to the specific embodiments thereof, it should be understood by those skilled in the art that various changes may be made and equivalents may be substituted to adapt the present invention to a particular situation. All such changes and modifications are within the scope of the present invention.

What is claimed is:

1. A method comprising:
a) obtaining a set of coordinates of a three dimensional structure of a protein TBM having n number of residues;
b) selecting a candidate residue i on the three dimensional structure of the TBM wherein the candidate residue i is the ith residue where i is a number between 1 and n and residue i is not a cysteine;
c) selecting a residue j where residue j is adjacent to residue i in sequence;
d) determining a candidate reference value wherein the candidate reference value is a spatial relationship between residue i and residue j;
e) obtaining a database comprising sets of coordinates of disulfide-containing protein fragments wherein each fragment comprises at least a disulfide-bonded cysteine and a first adjacent residue where the disulfide-bonded cysteine and the first adjacent residue share the same sequential relationship as residue i and residue j;
f) determining a comparative reference value for each fragment wherein the comparative reference value is the corresponding spatial relationship between the disulfide-bonded cysteine and the first adjacent residue as the candidate reference value is between residue i and j; and,
g) determining a score wherein the score is a measure of the number of fragments in the database that possess a comparative reference value that is the same or similar to the candidate reference value.

2. The method of claim 1 further comprising:
selecting a residue k where residue k is adjacent to residue i in sequence and k is not j; and
wherein
the candidate reference value is a spatial relationship between residue i, residue j, and residue k;
each fragment comprises at least a disulfide-bonded cysteine, a first adjacent residue, and a second adjacent residue where the disulfide-bonded cysteine and the first and second adjacent residues share the same sequential relationship as residue i, residue j, and residue k; and
the comparative reference value is the corresponding spatial relationship between the disulfide bonded cysteine, the first adjacent residue, and the second adjacent residue as the candidate reference value is between residue i, residue j, and residue k.

3. A method comprising:
a) obtaining a set of coordinates of a three dimensional structure of a protein TBM having n number of residues;
b) selecting a candidate residue i on the three dimensional structure of the TBM wherein the candidate residue i is the ith residue where i is a number between 1 and n and residue i is not a cysteine;
c) selecting residue j and residue k wherein residue j and residue k are both adjacent in sequence to residue i;
d) determining a candidate reference value wherein the candidate reference value is a spatial relationship of at least one backbone atom from each of residue i, residue j, and residue k;
e) obtaining a database comprising sets of coordinates of disulfide-containing protein fragments wherein each fragment comprises at least a disulfide-bonded cysteine, a first adjacent residue, and a second adjacent residue where the disulfide-bonded cysteine, the first adjacent residue, and the second adjacent residue share the same sequential relationship as residue i, residue j, and residue k;
f) determining a comparative reference value for each fragment wherein the comparative reference value is the corresponding spatial relationship between the disulfide-bonded cysteine, the first adjacent residue, and the second adjacent residue as the candidate reference value is between residue i, residue j, and residue k; and,
g) determining a score wherein the score is a measure of the number of fragments in the database that possess a comparative reference value that is the same or similar to the candidate reference value.

4. The method of any one of claims 1-3 wherein the spatial relationship comprises a dihedral angle.

5. The method of any one of claims 1-3 wherein the spatial relationship comprises a pair of phi psi angles.

6. The method of any one of claims 1-3 wherein the spatial relationship comprises a plurality of distances between atoms of two residues.

7. The method of any one of claims 1-3 wherein residue i is at least partially surface accessible.

8. The method of claim 7 wherein residue i has an accessible surface area of at least about 20 Å².

9. The method of any one of claims 1-3 wherein residue i does not participate in a hydrogen bond interaction with a backbone atom of the TBM.

10. A method comprising:
a) obtaining a three dimensional structure of a TBM having n number of residues and a site of interest;
b) selecting a candidate residue i that is at or near the site of interest wherein the candidate residue i is the ith residue where i is a number between 1 and n and residue i is not a cysteine;
c) generating a set of mutated TBM structures wherein each mutated TBM structure possesses a cysteine residue instead of residue i and wherein the cysteine residue is placed in a standard rotamer conformation; and,
d) evaluating the set of mutated TBM structures.

11. The method of claim 10 wherein the cysteine residue is capped with a S-methyl group.

12. The method of claim 10 wherein the standard rotamer conformation for cysteine comprises:
a chi1 angle selected from the group consisting of about 60°, about 180°, and about 300°; and
a chi2 angle selected from the group consisting of about 60°, about 120°, about 180°, about 270°, and about 300°.

13. The method of claim 10 wherein evaluation step comprises determining whether each rotamer conformation makes an unfavorable steric contact with the TBM.

14. The method of claim 10 wherein the evaluation step comprises a force field calculation.

15. The method of claim 11 wherein the evaluation step comprises determining whether each rotamer conformation places the methyl carbon of the S-methyl group closer to the site of interest than the C_{β}

16. A set of variant proteins, said proteins each being a mutated version of a TBM wherein a naturally occurring non-cysteine residue of the TBM is mutated into a cysteine.

17. The set of claim 16 comprising at least 3 cysteine mutants.

18. The set of claim 16 wherein one or more naturally occurring cysteines of the TBM is mutated to a non-cysteine residue.

19. The set of claim 16 wherein the TBM is a cell surface or soluble receptor.

20. The set of claim 16 wherein the TBM is a cytokine.

21. The set of claim 16 wherein the TBM is an enzyme.

22. The set of claim 16 wherein the TBM is selected from the group consisting of IL-2; IL-4; TNF-α; IL-1 receptor; caspase-3; PTP-1B; HIV integrase; BACE1; MEK-1; Cat-S; caspase-1; IL-13; CD40L; BAFF; P53; mdm2; bcl-x; bax; CDC25A; CD28; B7; C5A; AKT; CD45; HER2; GSK-3; alpha-E/beta-7; tissue factor; and Factor VII.
